SEQUENCE LISTING (5 sequences)

SEQ ID NO: 1 - DNA, 1821 bp, Phaseolus vulgaris
gtagacaaaatcccatcttttcctacataattcttctacagttaaccttcaaatcatattttcattattcacaaatatctagtcattcatacgaataaatatatatttttttcacatacaattatgataatatattaaaaagtgaactttaaatttaatttaatcttataaaatcaacttataaaatgagatttctacctacgattaataaaaataactttgatatcatattaaaaaataaactttaaacctaactcaactttataaaaccaatttataaaataaaatttacactcagttatgaattataaaatgaaatagtttttaggtgacgtggaatctccatccgattaatcaatatttgggtgatgttattgttattatagaaactaaaaacatgccaaataatttacaatatatagattcagttaaatcaattcagcttgtctccttgactaataaaaaaaaactttagactattattcagatttacacttcatctctcatgatatccctcaaagtgaatttcattcatggcaccatttatataatcaacaattttaaaaatatgcaaatttgtaccagtaaatgctttaatgtccctgataaacacaaaaaaaaaaaaattcatatttttttcttattaaataaagaagttcattgtaagagaaattaggatccttcaatagaaaatgtgttatttcctcatcaccagacaaaggggcaacagttaacaaaacaaatttatgtttcatttgagattaaggaaggtaaggaagaaaaaagattaaaaaaaatgtccttatctctttgtttctgtaataataatataagagacttaaacttttaatataataattgtaattaggttttctagtcatgagcaccactcagagacaagatttcaagaaaacaattttgttaaacatcttattagaaacttttagttaagtcttgaagttagaattaaacaaaaaaaagtacacacgagaaacacaataaacccactaccgtcaggttatcataaggatgaaatgttttgatatcattaaatataacacacacaaaaatacatctaattataacaatatatgttatacatatatttttgtaaaaacttagagtttttcaaaacattctaatacatgattagagtttatagaaatacaaatatttaaaaaatataattttaaaaaaacattctaaagtcattcagatcctctcacacctgtgtgatcatttagtcatgtatgtagtacaatcattgtagttcacaacagagtaaaataaataaggataaactagggaatatatataatatatacaattaaataaaaaagggaaaatcaaattagaatttttgattccccacatgacacaactcaccatgcacgctgccacctcagctccctcctctccacacatgtctcatgtcactttcgactttggttttttcactatgacacaactcgccatgcatgttgccacgtgagctccttcctcttcccatgatgacaccactgggcatgcatgctgccacctcagctcccacctcttctcattatgagcctactggccatgcacactgccacctcagcactcctctcacttcccattgctacctgccaaaccgcttctctccataaatatctatttaaatttaaactaattatttcatatacttttttgatgacgtggatgcattgccatcgttgtttaataattgttaatttggagttgaataataaaatgaaagaaaaaagttggaaagattttgcatttgttgttgtataaatagagaagagagtgatggttaatgca

SEQ ID NO: 2 - DNA, 13 bp, Phaseolus vulgaris
tgaatgcatgatc

SEQ ID NO: 3 - DNA, 1280 bp, Phaseolus vulgaris
actcccaaaaccaccttccctgtgacagttaaaccctgcttatacctttcctcctaataatgttcatctgtcacacaaactaaaataaataaaatgggagcaataaataaaatgggagctcatatatttacaccatttacactgtctattattcaccatgccaattattacttcataattttaaaattatgtcatttttaaaaattgcttaatgatggaaaggattattataagttaaaagtataacatagataaactaaccacaaaacaaatcaatataaactaacttactctcccatctaatttttatttaaatttctttacacttctcttccatttctatttctacaacattatttaacatttttattgtatttttcttactttctaactctattcatttcaaaaatcaatatatgtttatcaccacctctctaaaaaaaactttacaatcattggtccagaaaagttaaatcacgagatggtcattttagcattaaaacaacgattcttgtatcactatttttcagcatgtagtccattctcttcaaacaaagacagcggctatataatcgttgtgttatattcagtctaaaacaattgttatggtaaaagtcgtcattttacgcctttttaaaagatataaaatgacagttatggttaaaagtcatcatgttagatcctccttaaagatataaaatgacagttttggataaaaagtggtcattttatacgctcttgaaagatataaaacgacggttatggtaaaagctgccattttaaatgaaatatttttgttttagttcattttgtttaatgctaatcccatttaaattgacttgtacaattaaaactcacccacccagatacaatataaactaacttactctcacagctaagttttatttaaatttctttacacttcttttccatttctatttctatgacattaactaacatttttctcgtaattttttttcttattttctaactctatccatttcaaatcgatatatgtttatcaccaccactttaaaaagaaaatttacaatttctcgtgcaaaaaagctaaatcatgaccgtcattttagcattaaaacaacgattcttgtatcgttgtttttcagcatgtagtccattcttttcaagcaaagacaacagctatataatcatcgtgttatattcagtctaaaacaacagtaatgataaaagtcatcattttaggcctttctgaaatatatagaacgacattcatggtaaaaaatcgtcattttagatcc

SEQ ID NO: 4 - DNA, 63 bp, Arabidopsis thaliana
atggcaaacaagctcttcctcgtctgcgcaactttcgccctctgcttcctcctcaccaacgcc

SEQ ID NO: 5 - DNA, 1415 bp, Phaseolus vulgaris
ggtcgacggtatcgataagcttgatatcgaattcctgcagcccaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttggataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattactgtgggttgccatggcactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgca

(19) United States
(12) Patent Application Publication
(10) Pub. No.: US 2003/0159183 A1
(43) Pub. Date: Aug. 21, 2003

(54) Heterologous gene expression in plants

(76) Inventors: Geert Angenon, Gent (BE); Geert De Jaeger, Evergem (BE); Alain Goossens, Lokeren (BE); Anna Depicker, Merelbeke (BE)

Correspondence Address: TRASK BRITT, P.O. BOX 2550, SALT LAKE CITY, UT, 84110, US

(21) Appl. No.: 10/331,152

(22) Filed: Dec. 27, 2002

Related U.S. Application Data
(63) Continuation of application No. PCT/EP01/06298, filed on May 31, 2001.

(30) Foreign Application Priority Data
Jun. 29, 2000 (EP) 00202278.8

(51) Int. Cl. A01H 5/00; C12N 15/82

(52) U.S. Cl. 800/288; 435/468; 800/313

(57) ABSTRACT

The present invention relates to heterologous gene expression in plants. More specifically, the invention relates to high expression of heterologous proteins in seeds, by incorporating the gene between seed specific sequences. Preferably, the heterologous protein is a single-chain antibody variable fragment (scFv).

### CROSS-REFERENCE TO RELATED APPLICATION

This application is a continuation of International Application Number PCT/EP01/06298 filed on May 31, 2001 designating the United States of America, International Publication No. WO 02/00899 (Jan. 3, 2002), published in English, the contents of the entirety of which are incorporated by this reference.

### TECHNICAL FIELD

The present invention relates to heterologous gene expression in plants. More specifically, the invention relates to high expression of heterologous proteins in seeds, by incorporating the gene between seed specific sequences. Preferably, the heterologous protein is a single-chain antibody variable fragment (scFv).

### BACKGROUND

The ability to clone and produce a wide range of proteins from diverse sources became feasible with the advent of recombinant technology. The selection of expression hosts for commercial production of heterologous proteins is based on the economics of the production technique, such as the fermentation cost, on the cost of the purification and on the ability of the host to accomplish the post-translational modifications needed for full biological activity of the recombinant protein.

Although in many cases, prokaryotic cells such as Escherichia coli or simple eukaryotic cells such as the yeast Saccharomyces cerevisiae are the host cells of choice, these systems are not sufficient in all cases and problems can be encountered both in yield and activity of the protein produced. Alternative systems such as plant cells, mammalian cells and insect cells may solve the problem of biological activity, but suffer from a high fermentation cost and a low yield.

Transgenic plants can produce several types of heterologous polypeptides, comprising antibodies (ab's) and antibody fragments (Whitelam et al., 1993; Goddijn and Pen, 1995; Hemming, 1995). Antibodies and antibody fragments are interesting from an industrial point of view: they can be produced against nearly every type of organic molecule and bind the antigen in a very specific way. However, a major drawback is the production cost. Plants and plant cells are an interesting alternative for the production of these molecules, and other polypeptides that are difficult to produce in prokaryotic or other eukaryotic cells.

U.S. Pat. No. 5,804,694 describes the commercial production of β-glucuronidase in plants, by placing the β-glucuronidase gene after an ubiquitin promoter. With this construction, 0.1% of the total extracted protein is β-glucuronidase. By targeting the heterologous protein to the endoplasmic reticulum, the accumulation can be improved, especially for antibodies (ab's) and ab fragments. Use of the cauliflower mosaic virus 35S promoter resulted in expression of single chain Fv (scFv) antibodies, wherein up to 4-6.8% of the total soluble protein in leaves constituted scFv protein (Fiedler et al., 1997).

Much effort has been focused on the production of proteins in plant seeds. Indeed, seeds, especially those of legumes and cereals, contain large quantities of protein; these are mainly storage proteins, which can form up to 7-15% of the dry weight for cereals, and up to 20-40% for legumes. Moreover, those storage proteins are limited in number, and some of the individual storage proteins can be responsible for up to 20% of the total protein content of a seed (Vitale & Bollini, 1995), which may have important advantages for the purification process. Because of this, the promoters for the storage proteins have been considered as ideal tools to obtain high expression levels of heterologous proteins in seed (Fiedler el al., 1997).

U.S. Pat. No. 5,504,200 discloses the use of the phaseolin promoter for the expression of heterologous genes in plants and plant cells. PCT International Patent Publication WO 9113993 describes the expression of animal genes, or the gene from brazil nut 2S storage protein, using a promoter selected from the group consisting of the phaseolin promoter, the α′-subunit of β-conglycinin promoter and the β-zein promoter. The gene is linked to a poly-A signal selected from the group consisting of phaseolin poly-A signal and animal poly-A signal. However, none of these systems leads to high heterologous protein expression. PCT International Patent Publication WO9729200 describes a seed specific heterologous protein expression level of 1.9% of the total soluble protein, using the specific legumin B4 promoter. Further improvement of the expression cassettes lead to the production of scFv antibodies in ripe tobacco seeds constituting 3-4% of the total soluble protein (Fiedler et al., 1997).

Recently, the arcelin 5I gene (arc5I) of Phaseolus vulgaris was isolated and cloned. This gene is responsible for the production of the seed storage protein Arcelin 5a (ARC5a) that accumulates in wild type plants up to 24-32% of the total protein content of the seed (Goossens et al., 1994; Goossens et al., 1995). Expression of the arc5I gene in Arabidopsis thaliana and Phaseolus acutifolius indicated that the seed storage protein, ARC5a, could be expressed at levels up to 15% and 25%, respectively, of the total soluble protein (Goossens et al, 1999). However, no evidence was presented showing that the promoter could give efficient expression of other proteins.

### SUMMARY OF THE INVENTION

Surprisingly, we found that the use of the arcelin 5I promoter or the phaseolin promoter, in combination with the arcelin 5I leader sequence or the Tobacco Mosaic Virus (TMV) omega leader and the arcelin 5I3′ sequence results in an expression level of a heterologous protein as high as 12% of the total seed protein, which is far higher than known in the prior art.

It is a first aspect of the invention to provide a seed preferred expression cassette having gene regulatory elements comprising:

a) the arcelin promoter comprising the sequence shown in SEQ ID NO: 1 or a phaseolin promoter comprising SEQ ID NO: 5;

b) the arcelin 5I leader shown in SEQ ID 2 or a TMV omega leader; and

c) the arcelin 5I 3′ end comprising the sequence shown in SEQ ID NO: 3.

The seed preferred expression cassette may comprise the arcelin promoter, the arcelin 5I leader and the arcelin 5I 3′ end.

The seed preferred expression cassette may also comprise the sequence shown in SEQ ID NO: 4, encoding the 2S2 storage albumin signal peptide of Arabidopsis thaliana (Krebbers et al., 1988). In order to obtain seed specific expression of a gene of interest, the gene is placed between the leader sequence and the arcelin 3′ end sequence. Preferably, the gene of interest is fused to the sequence encoding the 2S2 storage albumin signal peptide. In one preferred embodiment, the gene of interest is a gene encoding a scFv antibody.

It is another aspect of the invention to provide a seed preferred expression cassette, which is not prone to silencing. When using the expression cassette according to the present invention to express a gene of interest, more than 40% of the transformed lines are not silenced and show a high expression, preferably more than 50% of the lines are not silenced, even more preferably more than 75% are not silenced.

It is another aspect of the invention to provide a method to obtain seed preferred expression of a heterologous protein at a level of at least 10%, preferably a level of at least 15%, 20%, 25%, 30%, 35% or 40% of the total soluble seed protein, with the proviso that the heterologous protein expressed is not an unmodified seed storage protein, such as Arcelin 5a. Preferably, the heterologous protein is not unmodified Arcelin, Phaseolin or Zein. In a preferred embodiment, a seed preferred expression cassette according to the present invention is used. Another preferred embodiment is a method according to the present invention, whereby the heterologous protein is a scFv.

Still another aspect of the present invention is a plant cell, transformed with an expression cassette according to the present invention, or a transgenic plant comprising an expression cassette according to the present invention. Indeed, the expression cassette can be incorporated and transformed into a plant cell or plant, using methods known to the person skilled in the art. The methods include, but are not limited to Agrobacterium T-DNA mediated transformation, particle bombardment, electroporation and direct DNA uptake.

### BRIEF DESCRIPTION OF THE DRAWINGS

FIG. 1: Overview of the T-DNA vectors for the evaluation of scFv production in seeds of Arabidopsis thaliana.

LB and RB=the left and right border of the T-DNA.

pVS1=plasmid insertion of Pseudomonas aeruginosa for vector stability and replication in Agrobacterium tumefaciens.

pBR=ori of replication in Escherichia coli.

NptIl=the selection marker, neomycin phosphotransferase II, under control of the nos-promoter and having ocs 3′-termination and poly-adenylation-signals.

Sm/SpR=a bacterial resistance gene for spectinomycin and streptomycin.

FIG. 2: Construction of pBluescript (2S2-G4).

FIG. 3: Construction of patag5 (3′-arc5I).

FIG. 4: Construction of pSP72 (Parc5I/ARC5a^{cs}).

FIG. 5: Amplification of DNA fragments for vector construction

FIG. 6: Construction of pParc5I-G4. This vector has been, in accordance with the Budapest Treaty, deposited with the Belgian Coordinated Collections of Microorganisms-BCCM™ Laboratorium voor Moleculaire Biologie-Plasmidencollectie (LMBP), Universiteit Gent, K. L. Ledeganckstraat 35, B-9000 Gent, Belgium by Dr. Ann Depicker, Molenstraat 61, 9820 Merelbeke, Belgium (work: K. L. Ledeganckstraat 35, 9000 Gent, Belgium) and has accession number: LMBP 4128.

FIG. 7: Construction of pParc5I-Ω-G4.

FIG. 8: Construction of pP35S-G4.

FIG. 9: Construction of pPβ-phas-G4.

FIG. 10: Results of scFv-G4 quantification in seed extracts by quantitative Western blot. 500 ng protein of A-, P-, and Ω-seed extracts and 2.5 μg protein of 35S- and Col. O-extracts were loaded on a 10% SDS-PAGE gel. G4 proteins were detected by monoclonal anti-c-myc antibody and anti-mouse antibody coupled to alkaline phosphatase. Col O=negative control.

FIG. 11: scFv-G4 quantification in seed extracts from floral dip transformants by quantitative Western blot. Results are shown for 10 segregating T2 seed stocks transformed with pParc5I-G4 (upper blot), and 4 segregating T2 seed stocks transformed with pPβ-phas-G4 (lower blot). We loaded 1 micrograms of seed protein in lanes A1, A7, F28, F31, F38, and F39, 1.5 microgram in lanes A3, A5, A8, A15, A22, and A42; and 2 micrograms in lanes A14 and A16 on a 10% SDS-PAGE gel. The G4 proteins, which are myc-tagged, were detected by monoclonal anti-c-myc antibody and anti-mouse antibody coupled to alkaline phosphatase. M=molecular weight marker.

FIG. 12: Coomassie blue stained SDS/page gel showing separated Arabidopsis seed proteins from transgenics F28 (lanes 3 and 7), F31 (lanes 4 and 8), F38 (lanes 5 and 9), F39 (lanes 6 and 10), transformed with pPβ-phas-G4 and from an untransformed control plant (lane 2). Lanes 3, 4, 5, and 6 contain 30 micrograms of total protein and lanes 7, 8, 9, and 10 contain 20 micrograms of total protein. The arrow indicates the recombinant scFv protein band. Lane 1 contains the molecular weight marker. On the basis of the coomassie stained protein bands in the separate lanes, Image master VDS software measured the following G4 concentrations for each lane: 9.9% for F28 (11.3% by Western blot), 15.4% for F31 (20.0% by Western blot), 16.0% for F38 (19.0% by Western blot), and 9.6% for F39 (12.0% by Western blot).

FIG. 13: Schematic representation of the ELISA-test used to analyse the antigen binding activity of ex planta-extracted and E. coli-extracted scFv-proteins. (1) Coating of microtiter well with the monoclonal antibody 9E10, which binds the c-myc-tag of the scFv; (2) co-incubation of scFv from seed or E. coli with an excess of the antigen dihydroflavonole-4-reductase (DFR) from Petunia hybrida; detection of bound DFR with (3) polyclonal antiserum against DFR from rabbit and (4) polyclonal anti-rabbit serum coupled to alkaline phosphatase (AP).

FIG. 14: Results of the analysis of antigen-binding activity of seed-and E. coli-extracted scFv proteins by ELISA. The ELISA test (FIG. 12) was performed with different G4-concentrations (X-axis) in presence or absence (controls) of DFR-antigen. Y-axis represents the ELISA-signal (ΔFU/min).

FIG. 15: Construction of patag6 (3′-arc5I).

FIG. 16: Construction of pParc5I-G4bis.

FIG. 17: scFv-G4 quantification in different seeds (3/2, 3/3, 3/4, and 3/5) from a single transgenic Phaseolus acutifolius plant. From each seed extract, we loaded 3 (lanes a) or 4 (lanes b) micrograms of seed protein on a 10% SDS-PAGE gel. G4 proteins, c-myc tagged, were detected by monoclonal anti-c-myc antibody and anti-mouse antibody coupled to alkaline phosphatase. M=molecular weight marker.

### DETAILED DESCRIPTION OF THE INVENTION

Definitions

The following definitions are set forth to illustrate and define the meaning and scope of various terms used to describe the invention herein.

Seed preferred expression means that the expression preferably takes place in the seed, but does not exclude expression in other organs of the plant.

Gene of interest as used here means the coding sequence of a gene, which it is desired to obtain seed preferred expression.

Leader as used here means the 5′ end untranslated sequence.

Signal peptide indicates the initial function of the peptide in the 2S2 albumin storage protein but does not necessarily imply that the peptide has the same function and is processed in the same way when it is fused to the gene of interest.

Heterologous protein refers to any protein that can be expressed in seed but which is not an unmodified seed storage protein. In contrast, modified seed storage proteins (specific mutants, fusion proteins, improved seed storage proteins, or the like) are part of the present invention and are thus included in the definition of a heterologous protein.

The invention is further explained by the use of the following illustrative examples.

### EXAMPLES

### Example 1

### Cloning of the T-DNA Vectors for the Evaluation of scFv Production, Under Control of the arc5I Expression Signals of Phaseolus vulgaris in Arabidopsis thaliana

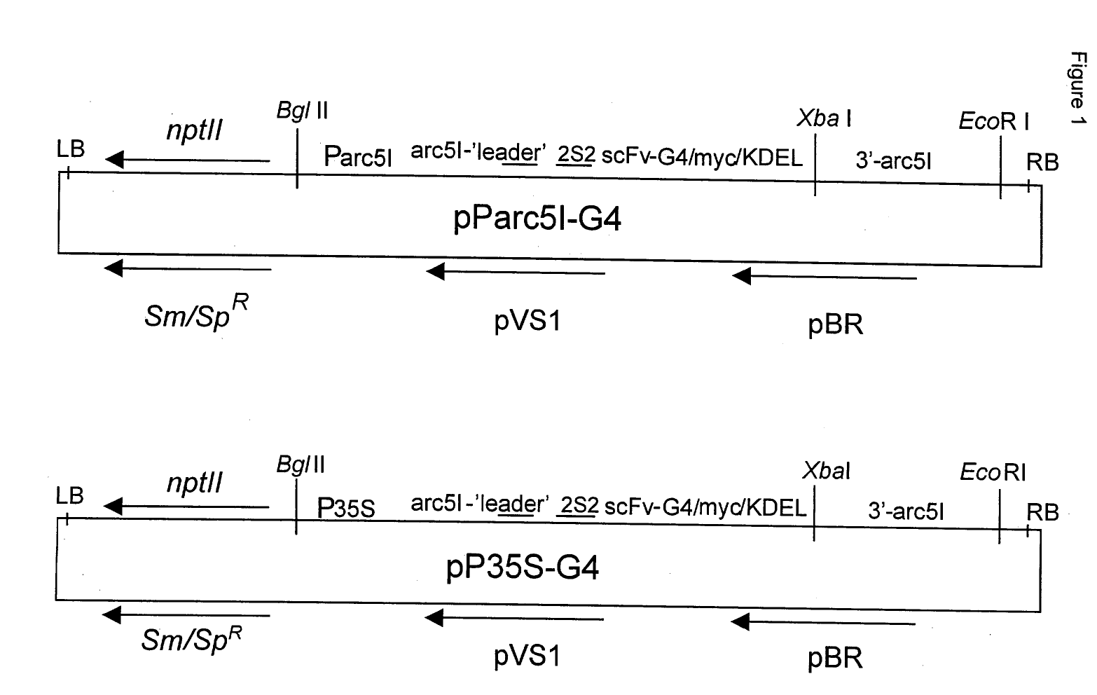

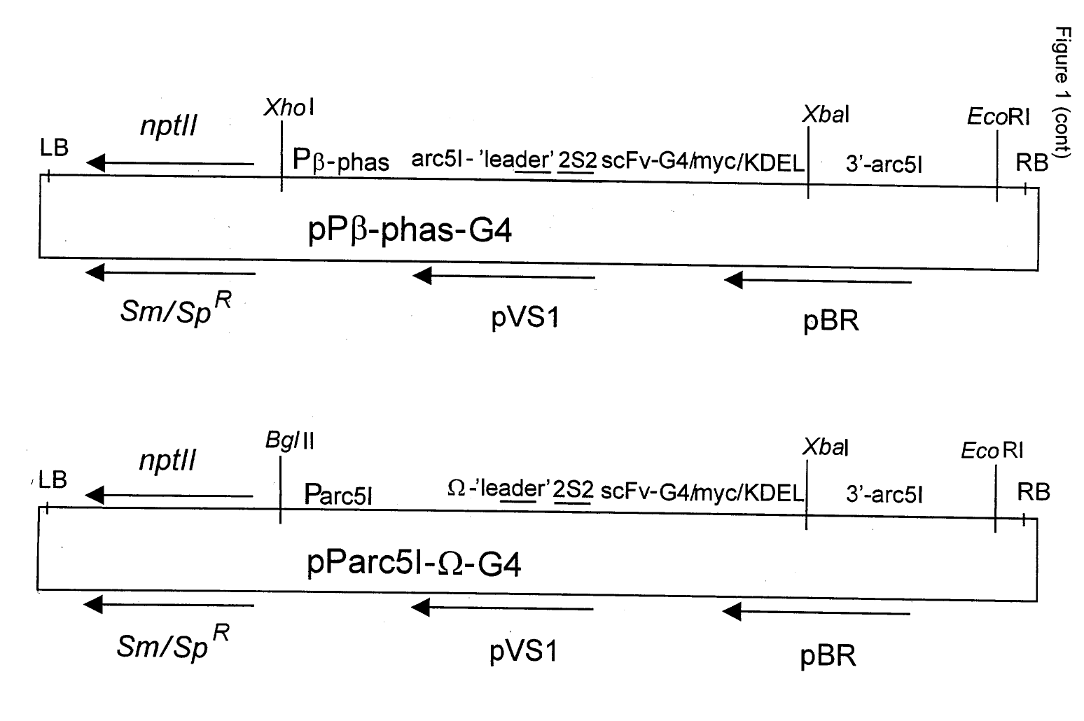

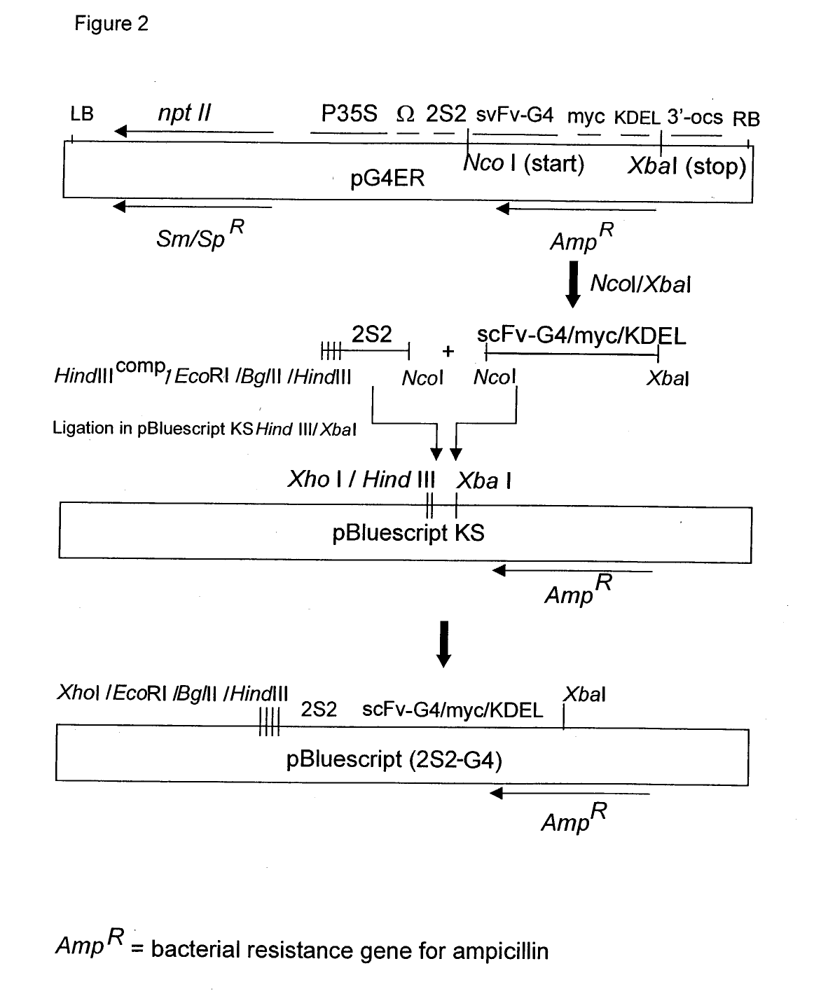

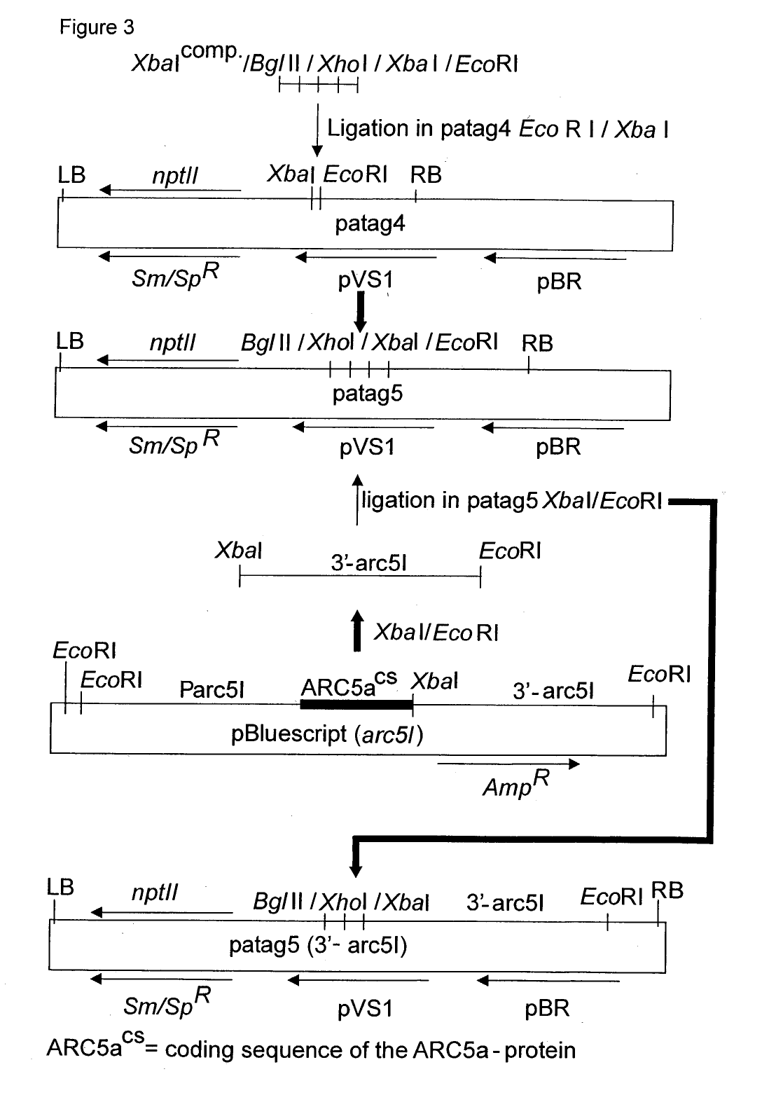

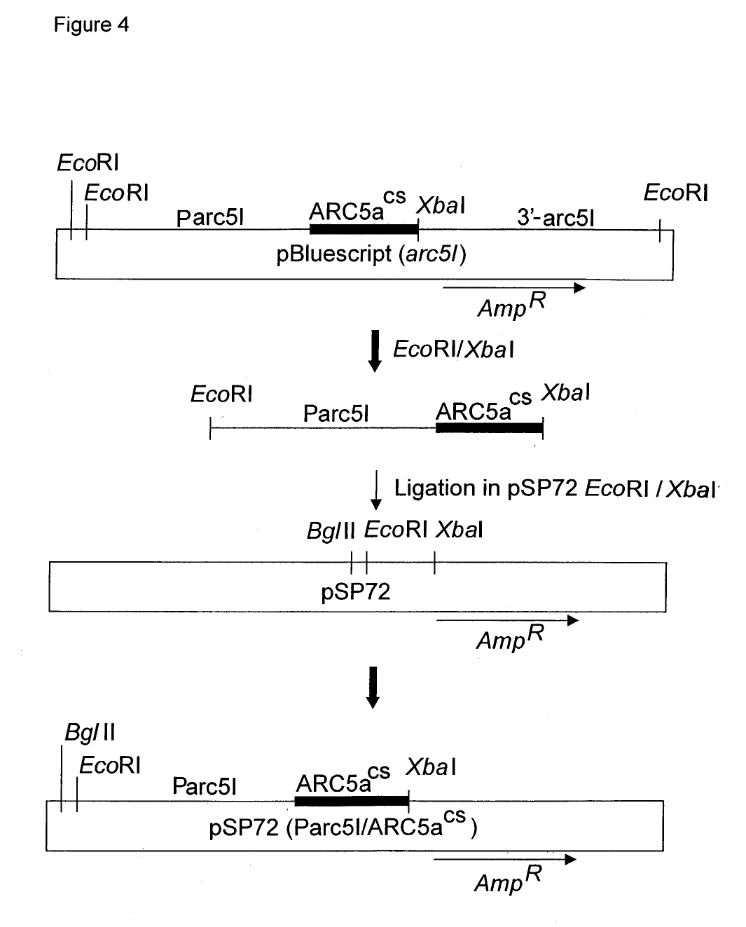

Four T-DNA vectors were constructed to evaluate and compare scFv production under control of the arc5I expression signals (vectors pParc5I-G4 and pParc5I-Ω-G4), the 35S promoter of the cauliflower mosaic virus (vector pP35S-G4), and the promoter of the β-phaseolin gene of Phaseolus vulgaris (vector pPβ-phas-G4) (FIG. 1). A first step in the cloning procedure consisted of the construction of three pilot vectors: pBluescript (2S2-G4) (FIG. 2), patag5 (3′-arc5I) (FIG. 3) and pSP72 (Parc5I/ARC5a^{cs}) (FIG. 4).

1.1 Construction of pBluescript (2S2-G4) (FIG. 2):

The coding sequence of the scFv fragment G4, fused at its 3′-end to the coding sequence of the c-myc tag (Evan et al., 1985) and the ER-retention signal KDEL (Denecke et al., 1992), was cut from the T-DNA vector pG4ER (De Jaeger et al., 1999) at the restriction sites Nco I and Xba I. In addition, a double stranded oligonucleotide was made, two appropriate single stranded oligonucleotides annealed together, that encodes the signal sequence of the seed storage protein 2S2 from Arabidopsis thaliana (Krebbers et al., 1988). This oligonucleotide was flanked at its 3′-end by the ‘sticky’ end, single stranded overhang, of the restriction site Nco I and was flanked at its 5′-end by a ‘sticky’ end that complements the overhang of a Hind III restriction site, but does not regenerate the site after ligation. The non-regenerating Hind III overhang is followed by the restriction sites for EcoR I, Bgl II and Hind III. The G4 fragment and the oligonucleotide were ligated together into the vector, pBluescriptKS (Stratagene, La Jolla, Calif.), which was digested with Hind III and Xba I. The sequence of the insert was checked and a clone containing a correct insert was selected for further cloning steps. As such, we obtained the pilot vector pBluescript (2S2-G4), which contained the G4-encoding sequence, preceded by a series of unique restriction sites that could be used for the insertion of the different promoters in combination with a mRNA-leader sequence.

1.2 Construction of patag5 (3′-arc5I) (FIG. 3):

An oligonucleotide was made to insert a few unique restriction sites in the T-DNA-vector patag4 (Goossens et al., 1999). The oligonucleotide contained the following restriction sites from its 5′-end to its 3′-end: the 5′-‘sticky’ end of Eco RI, the restriction sites Xba I, Xho I, and Bgl II and a 3′-‘sticky’ end that complements Xba I but does not regenerate the site after ligation. The oligonucleotide was ligated into patag4, digested with Eco RI en Xba I. This resulted in the vector patag5. The sequence of the inserted oligonucleotide was checked and a clone with the correct insert was selected for further cloning steps. From the vector pBluescript (arc5I) (Goossens et al., 1995), which contains the genomic sequence of the arc5I-gene, we cut out and isolated the 3′-expression signals of arc5I (3′-arc5I), using Xba I and Eco RI. The 3′-arc5I-fragment was then ligated into patag5, digested with Xba I and Eco RI. This resulted in the T-DNA-vector patag5 (3 ′-arc5I).

1.3 Construction of pSP72 (Parc5I/ARC5a^{cs}) (FIG. 4):

The arc5I promoter and the coding sequence of the ARC5a-protein (ARC5a^{cs}) were cut from pBluescript (arc5I), using Eco RI and Xba I. This fragment was ligated into the cloning vector pSP72 (Promega, Madison, Wis.), which was digested with the same restriction enzymes. This resulted in the vector pSP72 (Parc5I/ARC5a^{cs}), containing the arc5I-promoter preceded by the restriction sites Eco RI and Bgl II.

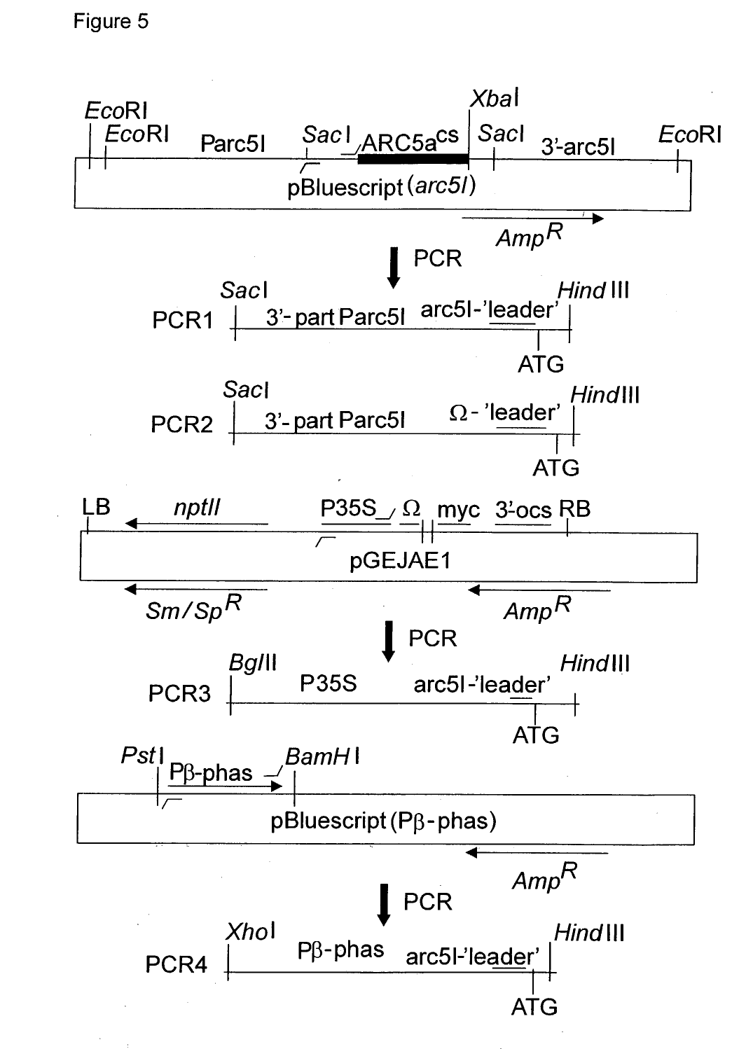

Besides these three pilot-vectors, four DNA fragments were amplified by PCR. These fragments were called PCR1, PCR2, PCR3 and PCR4 (FIG. 5). Fragments PCR1 and PCR2 were amplified from the 3′-end of the arc5I-promoter (Parc5I) in pBluescript (arc5I). PCR1 contained the 3′-end of Parc5I followed by the arc5I-‘leader’ and the 5′-end of the 2S2 signal sequence. PCR2 contained the same 3′-end of Parc5I, but is followed by the Ω-‘leader’ and the 5′-end of the 2S2 signal sequence. At the 3′-end of PCR1 and PCR2 (FIG. 5), we built into the appropriate primer the arc5I-‘leader’ followed by the 5′-end of the 2S2-signal sequence or the Ω-‘leader’ and the 5′-end of the 2S2 signal sequence, respectively. Both fragments were flanked by the restriction sites Sac I and Hind III. PCR3 was obtained by amplifying the 35S promoter of the cauliflower mosaic virus from the vector pGEJAE1 (De Jaeger et al., 1999). At the 3′-end of the 35S promoter, we built into the appropriate primer the arc5I-‘leader’, followed by the 5′-end of the 2S2 signal sequence. PCR3 is flanked by the restriction sites Bgl II and Hind III. PCR4 contained the promoter of the β-phaseolin gene of Phaseolus vulgaris, amplified from the vector pBluescript (Pβ-phas) (van der Geest et al., 1994). At the 3′-end of PCR4, we built into the appropriate primer the arc5I-‘leader’ followed by the 5′-end of the 2S2-signal sequence. This fragment was flanked by the restriction sites Xho I and Hind III. The pilot vectors and the four PCR fragments were used to clone the four T-DNA vectors from FIG. 1.

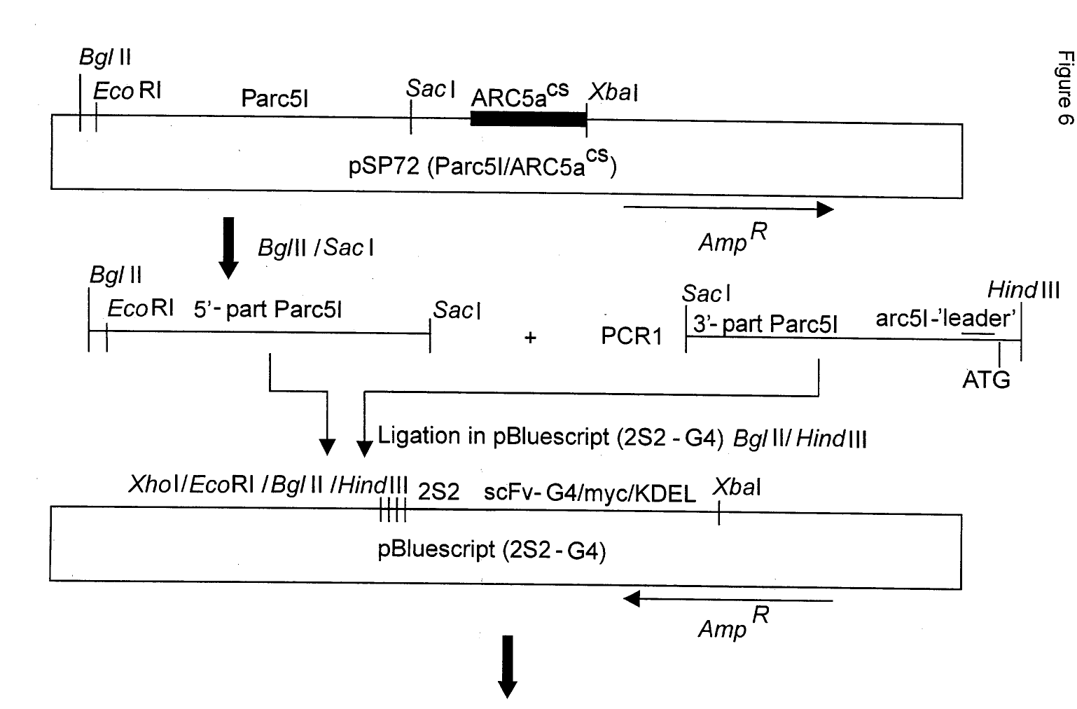

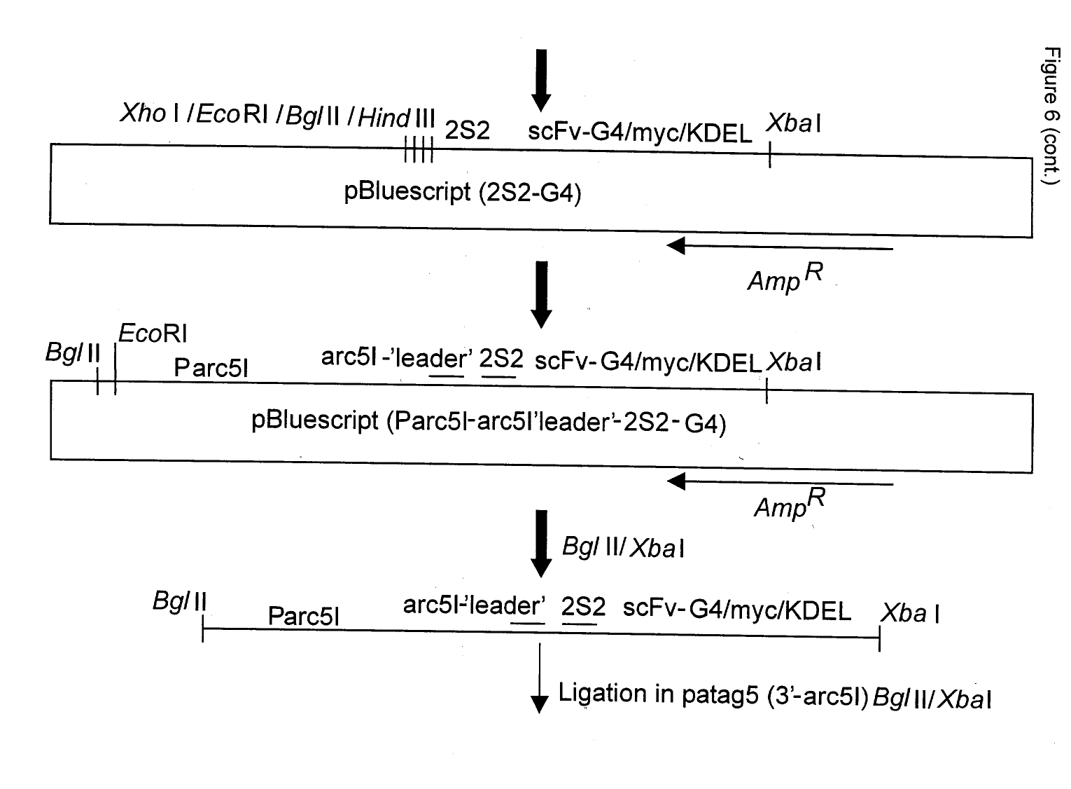

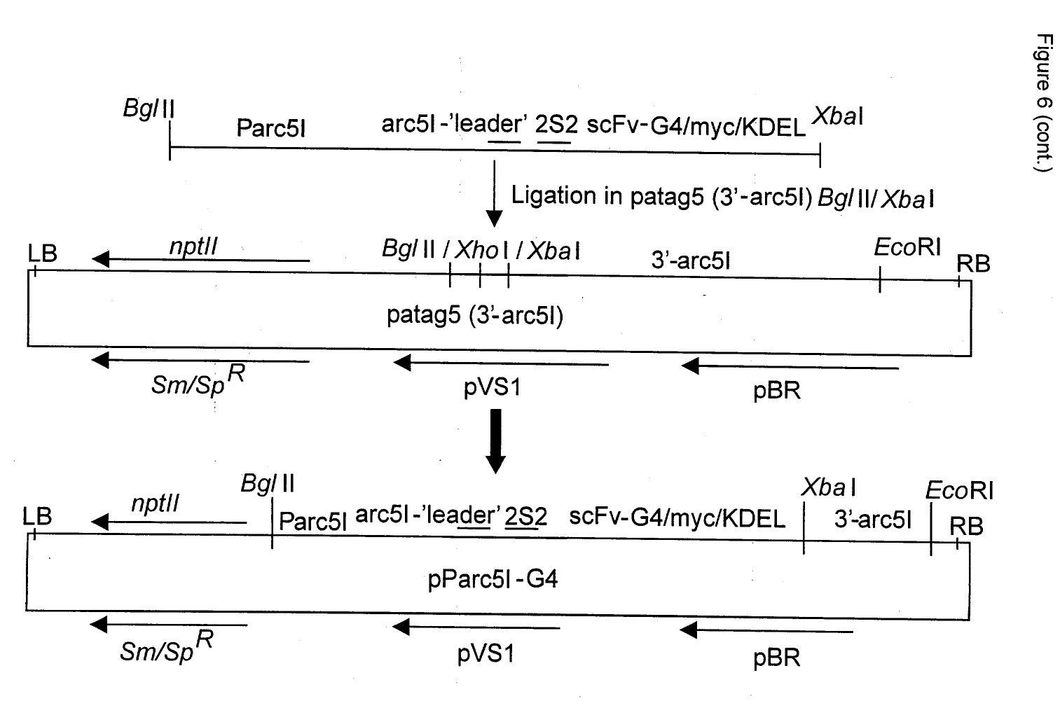

1.4 Construction of pParc5I-G4 (FIG. 6):

First, the 5′-end of the arc5I promoter was cut from the vector pSP72 (Parc5I/ARC5a^{cs}) by using Bgl II and Sac I enzymes. This promoter fragment, together with the PCR1 fragment, digested with Sac I and Hind III, were ligated into the Bgl II and Hind III digested vector pBluescript (2S2-G4). This resulted in the vector pBluescript (Parc5I-arc5I‘leader’-2S2-G4). The sequence of the inserted PCR1 fragment was checked and a clone with the correct insert was selected for further cloning steps. Finally, the arc5I-promoter with the arc5I-‘leader’ and the G4-coding sequence was cut from the former construct, (Parc5I-arc5I‘leader’-2S2-G4), using Bgl II and Xba I, which was then ligated in the Bgl II and Xba I digested vector patag5(3′-arc5I). This resulted in the T-DNA vector pParc5I-G4. This plasmid, transformed into E. coli MC1061 is deposited at BCCM under deposit number LMBP 4128. The plasmid contains the full arc5I-promoter and the full 3′end of arc5I, as used in the expression cassettes comprising the arc5I-promoter and the 3′end of arc5I.

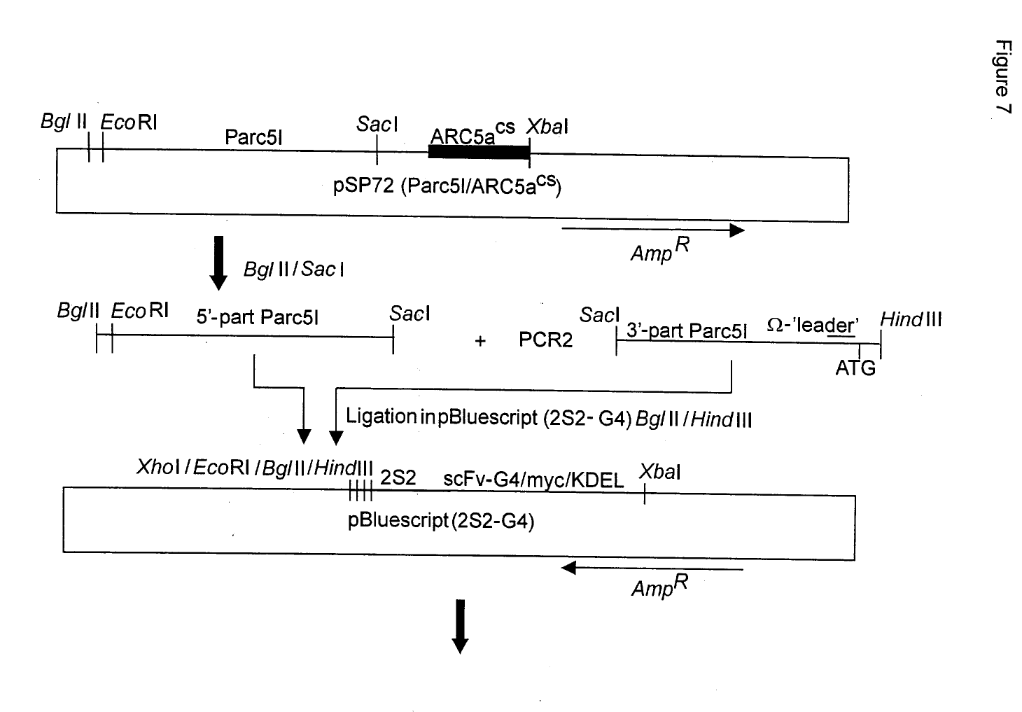

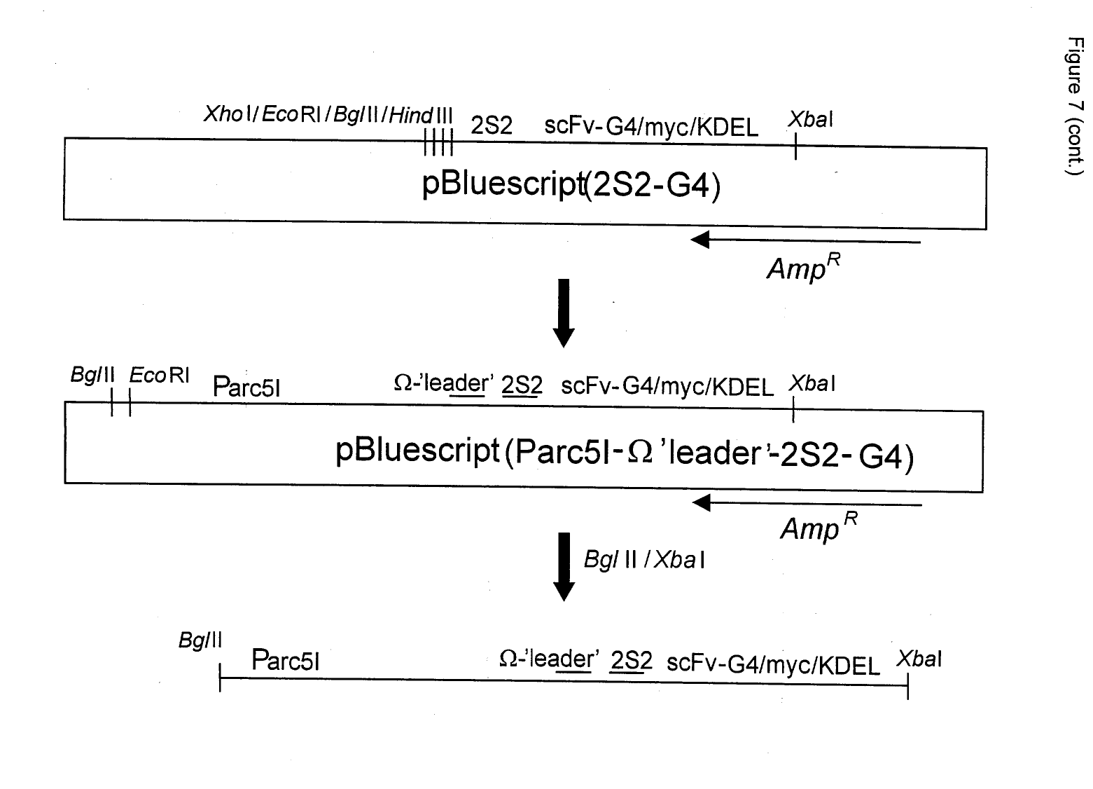

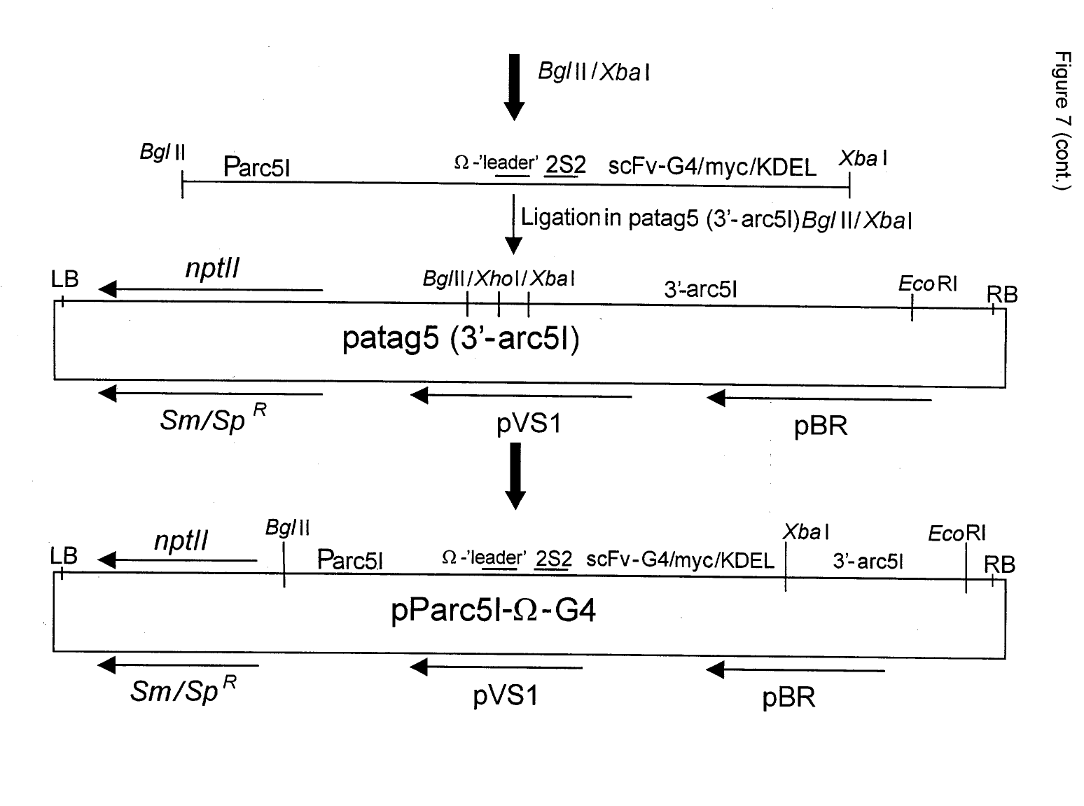

1.5 Construction of pParc5I-Ω-G4 (FIG. 7):

First, the 5′-end of the arc5I promoter was cut from the vector pSP72 (Parc5I/ARC5a^{cs}) by restriction digestion with Bgl TI and Sac I. This promoter fragment, together with the PCR2 fragment digested with Sac I and Hind III, was ligated into the vector pBluescript (2S2-G4) digested with Bgl II and Hind ITT. This resulted in the vector pBluescript (Parc5I-Ω‘leader’-2S2-G4). The sequence of the inserted PCR2 fragment was checked and a clone with the correct insert was selected for further cloning steps. Finally, the arc5I-promoter with the Ω-‘leader’ and the G4-coding sequence was cut from the former construct, (Parc5I-Ω‘leader’-2S2-G4), by digestion with Bgl IT and Xba I and ligated in the vector patag5 (3′-arc5I), which was digested with the same restriction enzymes. This resulted in the T-DNA vector pParc5I-Ω-G4.

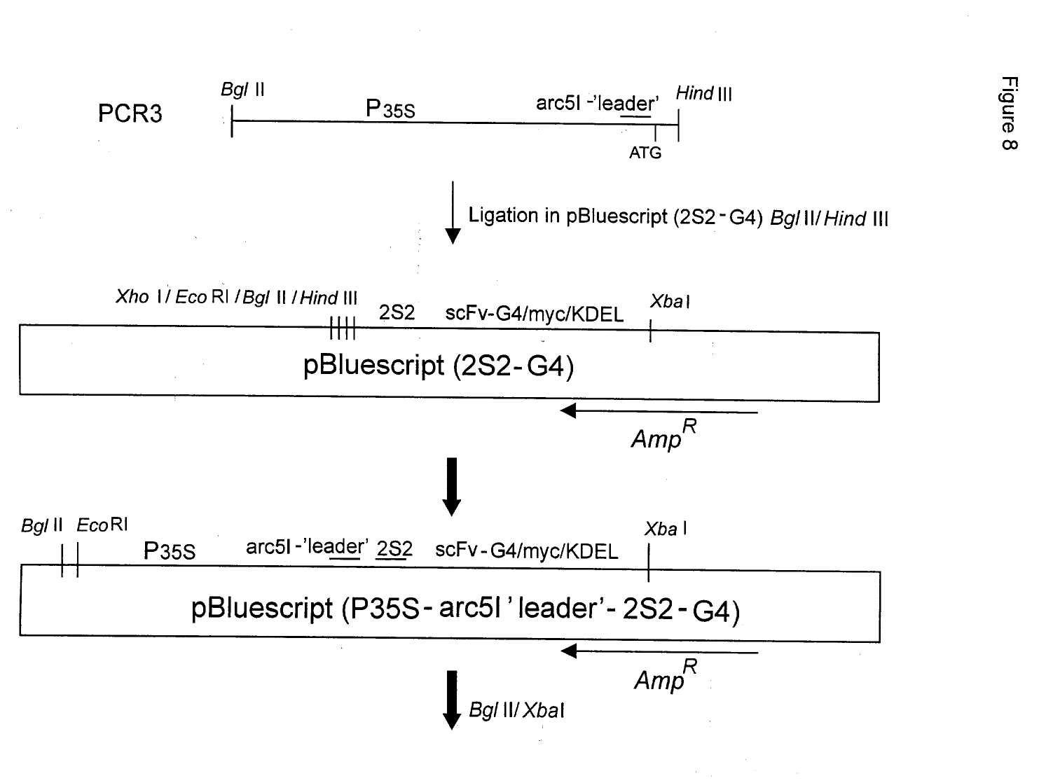

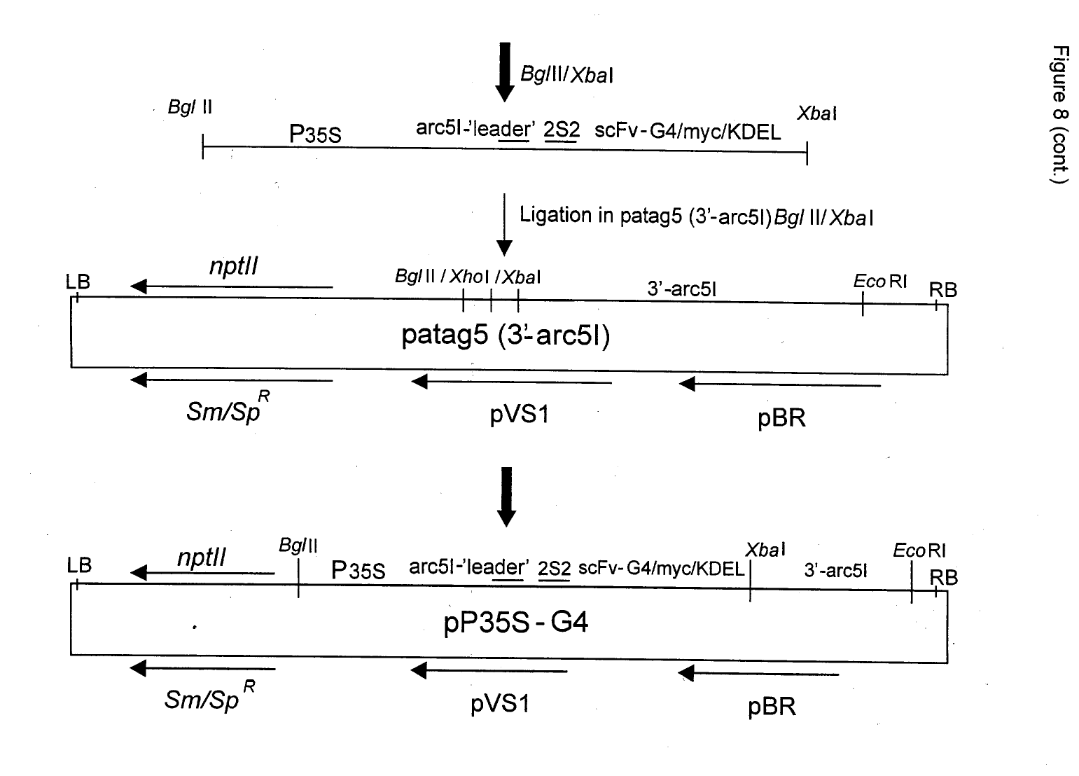

1.6 Construction of pP35S-G4 (FIG. 8):

Both PCR 3 and pBluescript (2S2-G4) were digested with Bgl II and Hind III, the PCR3 fragment was then ligated in the vector pBluescript (2S2-G4). This resulted in the vector pBluescript (P35S-arc5I‘leader’-2S2-G4). The sequence of the inserted PCR3 fragment was checked and a clone with the correct insert was selected for further cloning steps. Finally, the 35S-promoter with the arc5I-‘leader’ and the G4-coding sequence was cut from the former construct, (P35S-arc5I‘leader’-2S2-G4), by digestion with Bgl II and Xba I, then ligated in the vector patag5 (3′-arc5I) digested with the same restriction enzymes. This resulted in the T-DNA vector pP35S-G4.

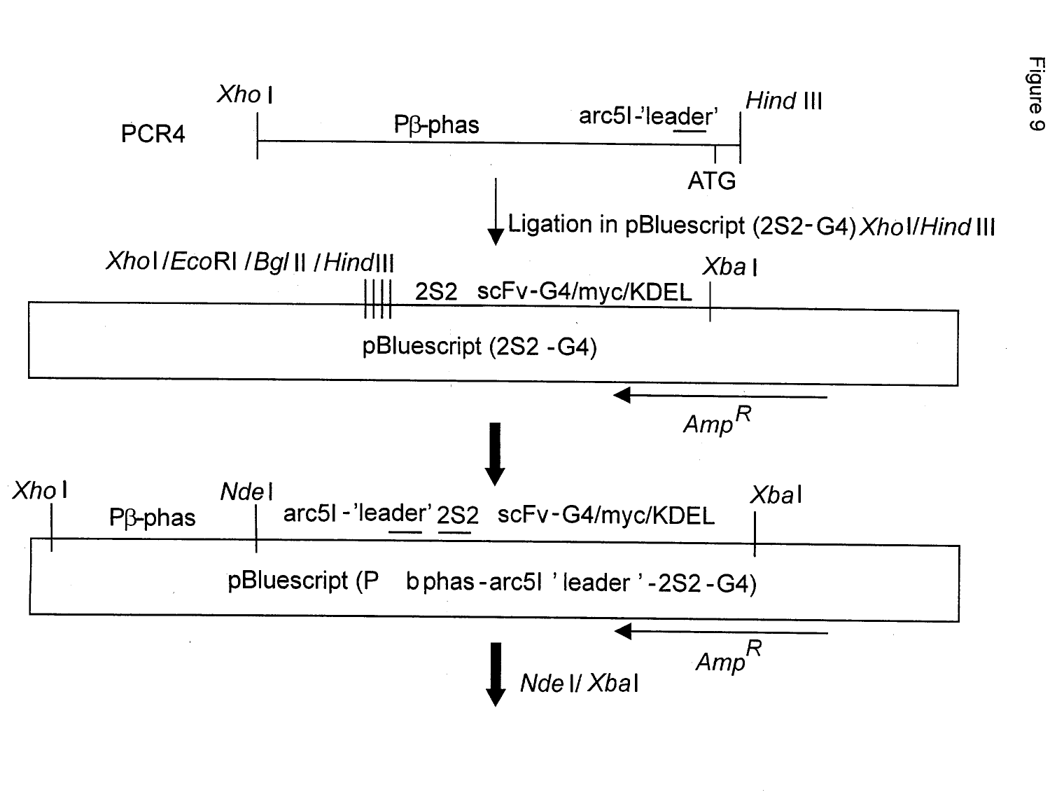

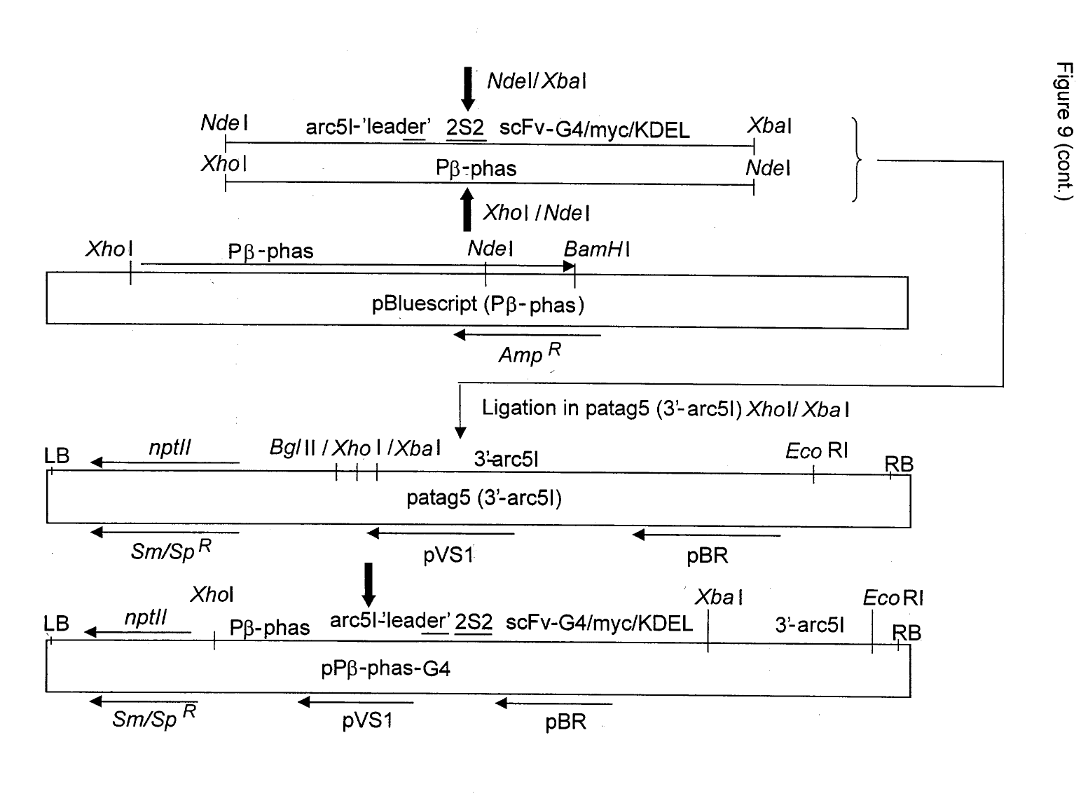

1.7 Construction of pPβ-phas-G4 (FIG. 9):

The PCR4 fragment was ligated in the vector pBluescript (2S2-G4), both digested with Xho I and Hind III. This resulted in the vector pBluescript (Pβphas-arc 5‘leader’-2S2-G4). After checking the DNA-sequence of the inserted PCR4-fragment, we found in the β-phaseolin promoter a few basepairs that differed from the original sequence (Bustos et al, 1991 ;Genbank accession number J01263). However, the 3′-end of the cloned promoter sequence, starting from the Nde I-site, was completely the same as the Genbank sequence. Therefore, the 3′ end of the β-phaseolin promoter, together with the coding sequence of scFv G4, was cut from the vector pBluescript (Pβphas-arc5I‘leader’-2S2-G4), using the restriction sites Nde I and Xba I. In addition, the 5′-end of the β-phaseoline-promoter was cut from pBluescript (Pβ-phas) at the restriction sites Xho T and Nde I. Both DNA-fragments were then ligated in patag5 (3′-arc 5I) digested with Xho I and Xba I. This resulted in the final vector pPβ-phas-G4. Again we checked the sequence of the β-phaseoline-promoter and the same base changes noted above were found. The sequence between the Xho I and Nde I sites, which contains the base changes and as used in the construct, is depicted in SEQ ID NO: 5.

The four T-DNA vectors were purified from Escherichia coli and electroporated into Agrobacterium tumefaciens C58C1Rif^{R} (pMP90). After colony purification, plasmids were purified from Agrobacterium and checked. The Agrobacterium-strains were used in subsequent Arabidopsis transformations.

### Example 2

### Transformation of Arabidopsis thaliana and Regeneration of Transgenic Plants

Arabidopsis thaliana (Columbia genotype O) was transformed by root-transformation (Valvekens et al., 1988) with the constructs pParc5I-G4, pParc5I-Ω-G4, pP35S-G4 and pPβ-phas-G4. After selection of transformed calli on kanamycin-selective medium, 150 calli were transferred to shoot inducing medium. Finally, shoots were transferred to root inducing medium. After root formation, plants were transfered to the greenhouse and seeds were collected from the following numbers of transgenic Arabidopsis plants: 36 for pParc5I-G4, 4 for pP35S-G4, 18 for pPβ-phas-G4, and 13 for pParc5I-Ω-G4.

In parallel, the same constructs were used for Arabidopsis transformation by ‘floral dip’ (Clough & Bent, 1998). Transformed T1-plants were selected on kanamycin-containing selective medium, transferred to the greenhouse, and seeds were collected.

### Example 3

### scFv Accumulation in Transgenic Arabidopsis Seeds

3.1 Seed Extraction and Protein Quantification:

Crude seed protein extracts were obtained following a modification of the extraction protocol of van der Klei et al. (1995) (Goossens et al., 1999). Ground seeds were extracted twice with hexane to remove lipids. The dried dilipidated powder was resuspended and extracted twice with 50 mM Tris/HCl, 200 mM NaCl, 5 mM EDTA, 0.1% Tween 20, pH 8 (Fiedler et al., 1997) for 15 min at room temperature under continuous shaking. To prevent protein degradation, a protease inhibitor mix (2× CØmplete™, Roche Molecular Biochemicals, Germany) was added to the extraction buffer. The pellets were removed by centrifugation at 20000 g and the supernatants were pooled. Total protein quantity in the crude extracts was determined by the Lowry method using the DC Protein Assay (BioRad, Hercules, USA) with BSA as a standard (Table 1). The reliability of Arabidopsis seed protein quantification by this method was proven by Goossens et al., 1999.

Table 1 shows total protein concentration in extracts of transgenic Arabidopsis seeds.

TABLE 1
Total protein concentration in transgenic seed extracts (500 microliters)
from 10 mg of transgenic Arabidopsis seeds.

| | | | | |
|---|---|---|---|---|
| A¹ 1 | A 105 | A 140 | A 143 | A 165A |
| 3,665 μg/μl | 3,395 μg/μl | 3,042 μg/μl | 3,708 μg/μl | 3,675 μg/μl |
| P² 3A | P 5 | P 15 | P 22A | P 102B |
| 2,852 μg/μl | 4,028 μg/μl | 3,623 μg/μl | 3,151 μg/μl | 3,339 μg/μl |
| Ω³ 7A | Ω 33 | Ω 65C | Ω 98A | Ω 130 |
| 3,873 μg/μl | 3,527 μg/μl | 3,184 μg/μl | 3,754 μg/μl | 3,517 μg/μl |
| 35S⁴ 93 | 35S 101 | 35S 116 | 35S 131A | Col O⁵ |
| 3,945 μg/μl | 3,837 μg/μl | 3,879 μg/μl | 3,906 μg/μl | 3,215 μg/μl |

¹A = a pParc5I-G4 transformed Arabidopsis line.
²P = a pPβ-phas-G4 transformed Arabidopsis line.
³Ω = a pParc5I-Ω-G4 transformed Arabidopsis line.
⁴35S = a pP35S-G4 transformed Arabidopsis line.
⁵Col O = a non-transformed Columbia genotype O used as a control.

3.2 Quantification of scFv-G4 Accumulation:

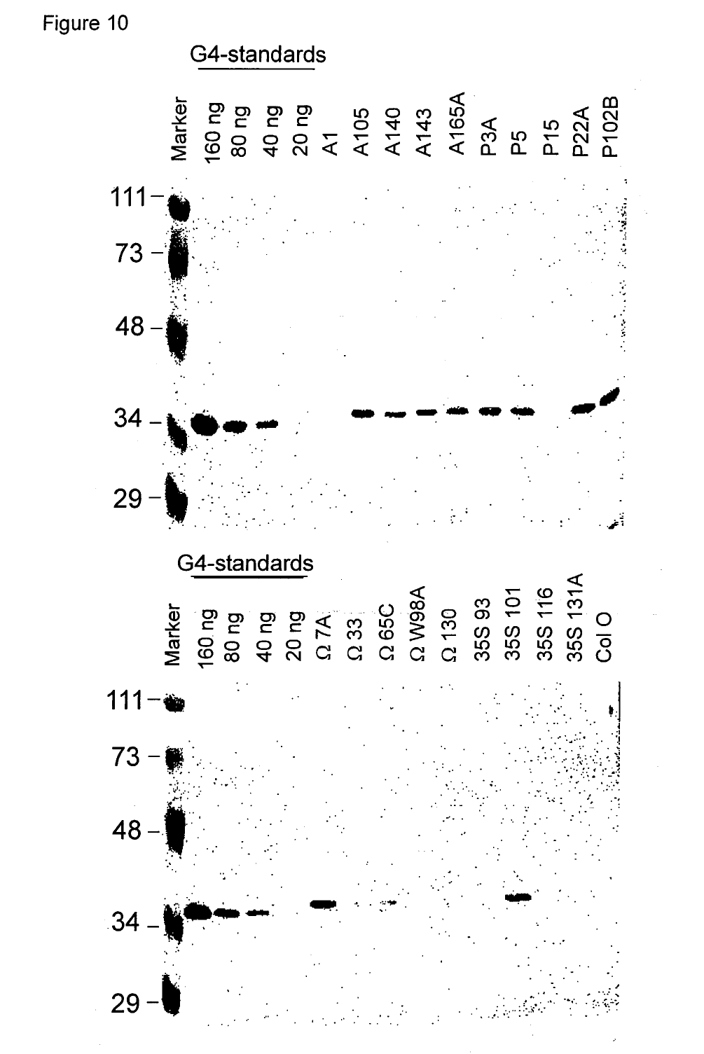

Total protein was separated on SDS/PAGE and accumulation levels of the scFv-G4 protein was determined by quantitative Western blot analysis using the anti-c-myc monoclonal antibody 9E10 (Evan et al., 1995) followed by anti-mouse IgG coupled to alkaline phosphatase (Sigma, St Louis, Mo., USA), according to De Jaeger et al., 1999 (FIG. 10). Different amounts of scFv-G4 proteins purified from Escherichia coli (De Jaeger et al., 1999) were used as standards. The constructs pParc5I-G4, pParc5I-Ω-G4, and pPβ-phas-G4 give very high scFv-G4 accumulation levels in Arabidospsis seeds, in the range of 10% of total soluble seed protein. These are the highest levels ever reported for scFv proteins produced in plants. Moreover, lines with such high levels were easily found, as only 5 lines were screened for each construct, which is an indication that the expression cassettes are not very sensitive to silencing.

Table 2 shows ScFv-G4 protein accumulation levels in transgenic Arabidopsis seeds.

TABLE 2
ScFv-G4 accumulation levels in transgenic Arabidopsis seeds.

| PParc5I-G4 | | pPβ-phas-G4 | | pParc5I-Ω-G4 | | pP35S-G4 | |
|---|---|---|---|---|---|---|---|
| | G4-Level | | G4-Level | | G4-Level | | G4-Level |
| Line | (*) | Line | (*) | Line | (*) | Line | (*) |
| A 1 | <4% | P 3A | 10% | Ω 7A | 12% | 35S 93 | <0.8% |
| A 105 | 10% | P 5 | 10% | Ω 33 | <4% | 35S 101 | 3% |
| A 140 | 6% | P 15 | <4% | Ω 65C | 6% | 35S 116 | <0.8% |
| A 143 | 8% | P 22A | 12% | Ω 98A | <4% | 35S | <0.8% |
| | | | | | | 131A | |
| A 165A | 8% | P 102B | 12% | Ω 130 | <4% | | |

(*) accumulation level as % of total soluble protein content in transgenic seeds

### Example 4

### Transformation of Arabidopsis thaliana by ‘Floral Dip’ and Regeneration of Transgenic Plants

Arabidopsis thaliana (Columbia genotype O) was transformed by ‘floral dip’ (Clough & Bent, 1998) with the constructs pParc5I-G4, pParc5I-Ω-G4, pP35S-G4, and pPβ-phas-G4. Transformed T1-plants were selected on kanamycin-containing selective medium, transferred to the greenhouse, and T2-segregating seed stocks were collected.

ScFv Accumulation in Transgenic T2-Segregating Seed Stocks

4.1 Seed Extraction and Protein Quantification:

Seed extraction and protein quantification was determined as described for Example 3.

4.2 Quantification of scFv-G4 Accumulation:

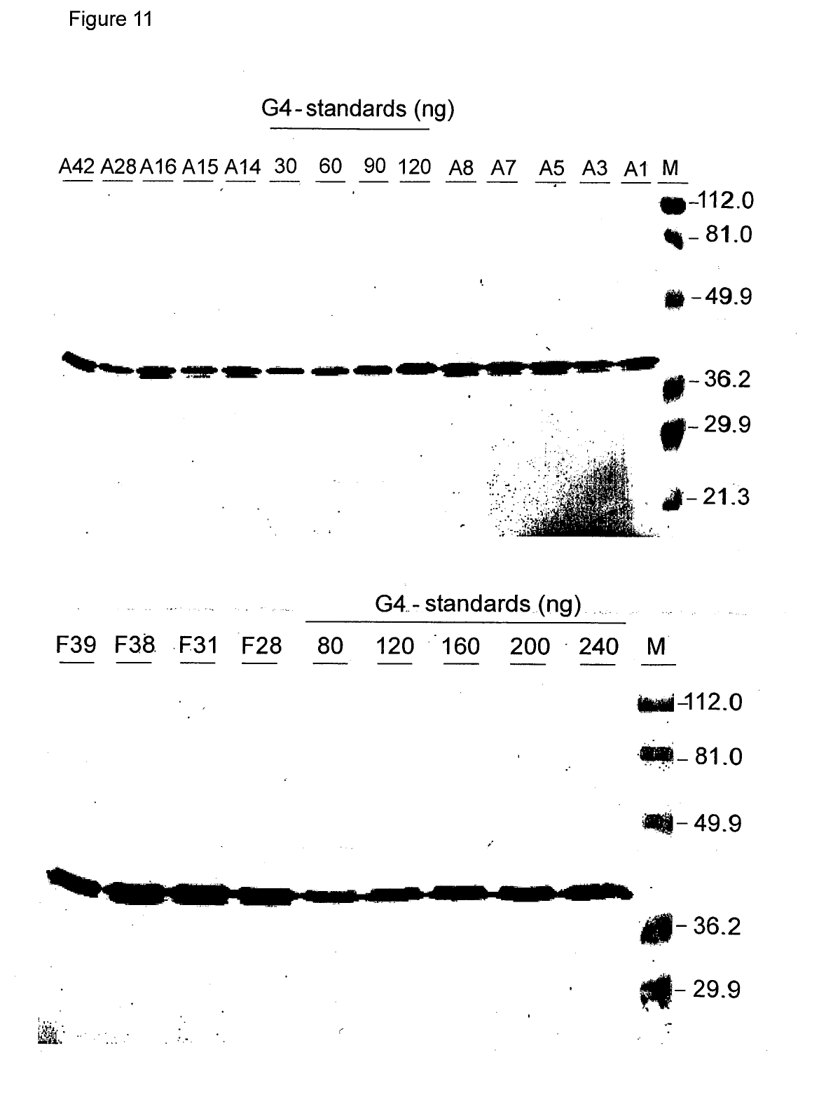

Total protein was separated on SDS/PAGE and accumulation levels of the scFv-G4 protein was determined by quantitative Western blot analysis using the anti-c-myc monoclonal antibody 9E10 (Evan et al., 1995) followed by anti-mouse IgG coupled to alkaline phosphatase (Sigma, St Louis, Mo., USA), according to De Jaeger et al., 1999 (FIG. 11). Different amounts of scFv-G4KDEL proteins purified from Escherichia coli were used as standards. Most seed stocks were analyzed at least two times. The constructs pParc5I-G4 and pPβ-phas-G4 give very high scFv-G4 accumulation levels in Arabidopsis seeds, in the range of 5-10% and 10%-20% of total soluble seed protein, respectively (table 3). Use of the arc5I-untranslated leader in pParc5I-G4 or the TMV(omega)-leader in pParc5I-Ω-G4 give similar accumulation levels (Table 3), showing that both leaders allow efficient translation initiation in seeds. In addition, inter-transgenic variation is low for all four constructs, which is an indication that the expression cassettes are not very sensitive to silencing. Seed extracts of four pPβ-phas-G4 plant lines with the highest G4-accumulation were further analysed by SDS/PAGE and Coomassie-blue staining (FIG. 12). A clear scFv-protein band could be identified at the expected size, which was absent in the untransformed control line. By using the Imagemaster VDS software (Pharmacia, Uppsala, Sweden), the percentage of scFv-protein relative to total soluble seed protein was measured in each lane. Similar scFv-accumulation levels were obtained using this method as were found in the same lines using the quantitative Western blot analysis.

The following table shows ScFv-G4 protein accumulation levels in transgenic Arabidopsis segregating T2-seed stocks.

TABLE 3
ScFv-G4 accumulation levels in transgenic Arabidopsis
segregating T2-seed stocks. 20 independent seed stocks were
analysed, scFv levels were ranked from highest to lowest.

| pParc5I-G4 (*) | pPβ-phas-G4 (*) | pParc5I-Ω-G4 (*) | pP35S-G4 (*) |
|---|---|---|---|
| 8.0 ± 1.4 | 20.0 ± 0.0 | 5.8 ± 0.4 | 1.1 ± 0.1 |
| 7.8 ± 0.4 | 19.0 ± 1.4 | 4.9 ± 0.2 | 1.1 ± 0.1 |
| 6.7 ± 0.1 | 12.0 ± 0.0 | 4.5 ± 0.7 | 1.1 ± 0.1 |
| 6.4 ± 0.5 | 11.3 ± 1.8 | 4.0 ± 0.0 | 1.0 ± 0.0 |
| 5.3 ± 1.8 | 7.2 ± 0.2 | 3.9 ± 0.2 | 1.0 ± 0.0 |
| 4.7 ± 0.5 | 5.4 ± 0.9 | 3.9 ± 0.2 | 0.8 ± 0.1 |
| 4.5 ± 0.7 | 5.0 ± 0.0 | 3.5 ± 0.2 | 0.7 ± 0.1 |
| 3.9 ± 0.2 | 4.5 ± 0.7 | 3.2 ± 0.2 | 0.7 ± 0.1 |
| 3.8 ± 0.4 | 4.4 ± 0.5 | 3.1 ± 0.6 | 0.7 ± 0.1 |
| 3.7 ± 0.5 | 4.0 ± 1.0 | 3.0 ± 0.0 | 0.7 ± 0.0 |
| 2.4 ± 0.5 | 3.9 ± 0.2 | 3.0 ± 0.0 | 0.7 ± 0.1 |
| 1.7 ± 0.5 | 3.8 ± 0.3 | 2.7 ± 0.4 | 0.7 ± 0.1 |
| 1.3 ± 0.4 | 1.8 ± 0.4 | 1.6 ± 0.4 | 0.6 ± 0.0 |
| 0.9 ± 0.2 | 1.8 ± 0.3 | 1.5 | 0.5 ± 0.1 |
| 0.8 ± 0.0 | 1.6 | 1.4 ± 0.1 | 0.5 ± 0.0 |
| 0.5 ± 0.1 | 1.3 | 0.7 ± 0.0 | 0.4 ± 0.0 |
| 0.5 ± 0.1 | 1.2 | 0.6 ± 0.1 | 0.3 ± 0.0 |
| 0.4 ± 0.1 | 0.8 | 0.3 | 0.2 ± 0.1 |
| <0.3 | <0.4 | 0.2 | 0.1 |
| <0.3 | n.d. | 0.1 | n.d. |

(*) scFv accumulation level as % of total soluble protein content in transgenic seeds, with standard deviation.
n.d. = not detectable.

ScFv Accumulation in Transgenic T3-Homozygous Seed Stocks

For each construct, 10 T2-seed stocks containing the highest G4-levels were genetically screened by segregation analysis. 72 seeds from each seed stock were germinated on kanamycin-containing selective medium and by statistical analysis we identified plant lines containing a single T-DNA locus. For the constructs pParc5I-G4 and pPβ-phas-G4, 4 lines containing a single T-DNA locus, were chosen to grow and select homozygous seed stocks. Ten T2-plants per line were grown, T3-seeds were collected and homozygous T3-seed stocks were selected using statistical analysis by growing plants on kanamycin-containing selective medium. For one of the four lines containing construct pParc5I-G4, no homozygous seed stocks were found. G4-accumulation was measured by quantitative Western blot in T3-segregating and T3-homozygous seed stocks.

Most pParc5I-G4 and all pPβ-phas-G4 homozygous seed stocks gave higher G4-levels than the corresponding T3-segregating stocks (Table 4). For both constructs pParc5I-G4 and pPβ-phas-G4, homozygous seed stocks were obtained, which contain the G4 protein as more than 10% of the total soluble seed protein. Homozygous seed stocks, transformed with pPβ-phas-G4, contained extraordinary high G4 levels, up to 36.5% of the total soluble protein in seeds. This is the highest heterologous protein level ever reported for plants.

Table 4 shows the accumulation of scFv-G4 protein in segregating and homozygous T3-seed stocks.

TABLE 4
ScFv-G4 accumulation levels in transgenic
Arabidopsis segregating and homozygous T3-seed stocks.

| | T2-segregating | T3-segregating | T3-homozygous |
|---|---|---|---|
| Plant line | seed stocks (*) | seed stocks (*) | seed stocks (*) |
| A1 | 8.0 ± 1.4 | 8.2 ± 1.0 | 4.4 ± 0.8 |
| | | 6.7 ± 0.7 | 12.5 ± 1.9 |
| A15 | 4.7 ± 0.5 | 5.6 ± 1.0 | 6.4 ± 1.4 |
| | | 4.7 ± 0.0 | 7.7 ± 1.4 |
| A16 | 4.5 ± 0.7 | 3.8 ± 0.7 | 6.0 ± 1.0 |
| | | 5.1 ± 1.4 | 3.5 ± 0.4 |
| F3 | 4.5 ± 0.7 | 5.8 | 18.0 |
| F24 | 5.0 ± 0.0 | 7.0 ± 1.4 | 13.5 ± 2.1 |
| | | 4.9 ± 0.2 | 15.0 ± 1.4 |
| F28 | 11.3 ± 1.8 | 13.0 ± 1.4 | 21.0 ± 4.2 |
| | | 10.5 ± 0.7 | 18.5 ± 0.7 |
| F38 | 19.0 ± 1.4 | 17.5 ± 0.7 | 36.5 ± 3.4 |
| | | 17.5 ± 3.5 | |

(*) scFv accumulation level as % of total soluble protein content in transgenic seeds, with standard deviation. For most lines more than one T3-segregating and homozygous seed stock was analysed.
n.d. = not detectable.
Lines A1, A15, and A16 are plant lines transformed with construct pParc5I-G4.
Lines F3, F24, F28, and F38 were transformed with pPβ-phas-G4.

Analysis of scFv-Quality in Seed Extracts

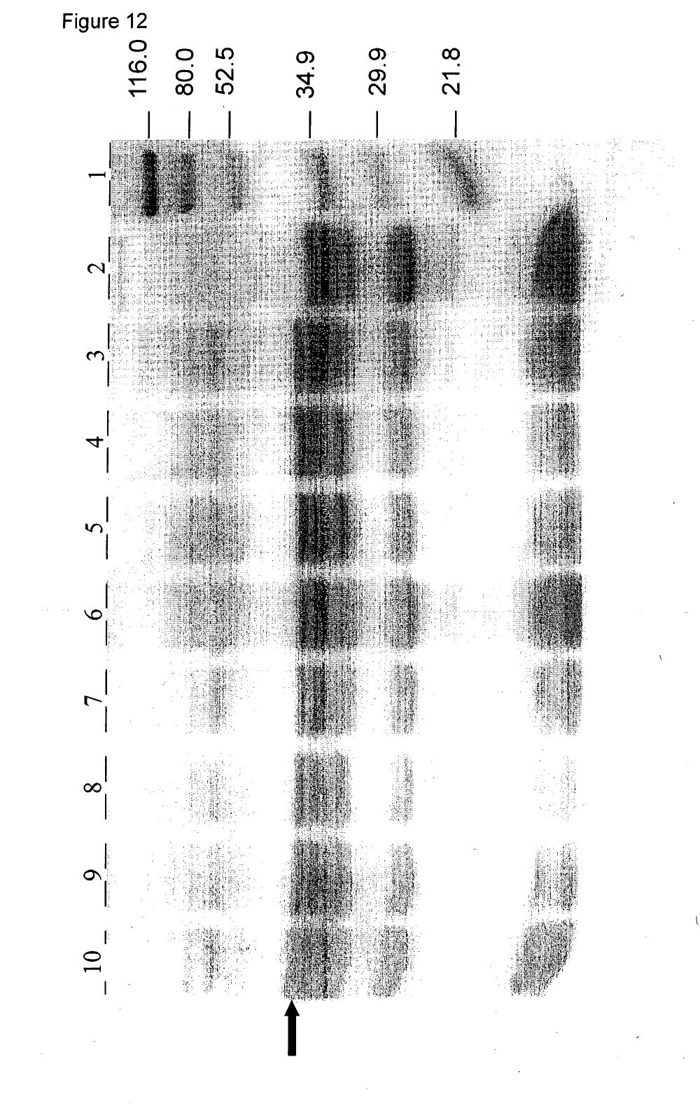

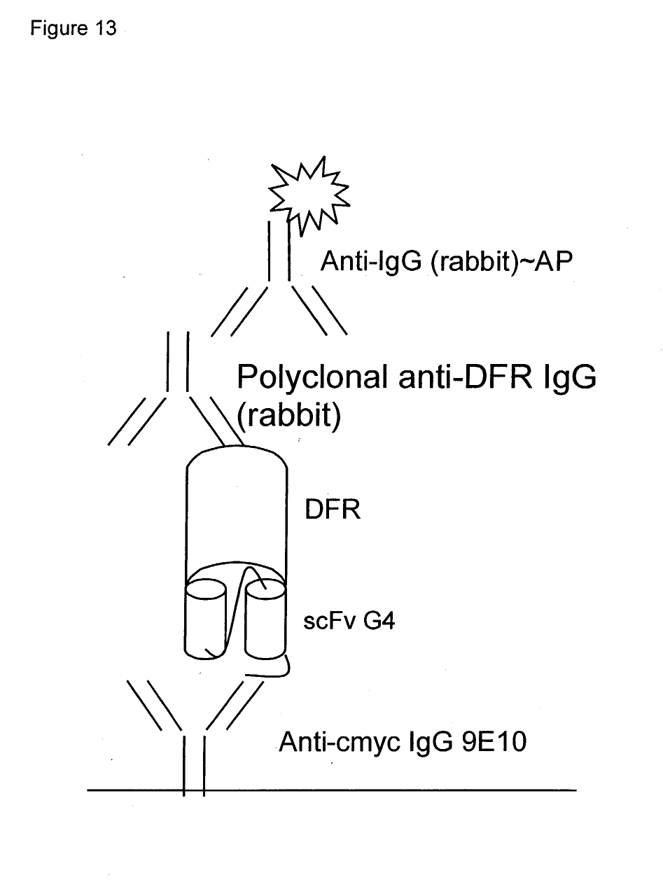

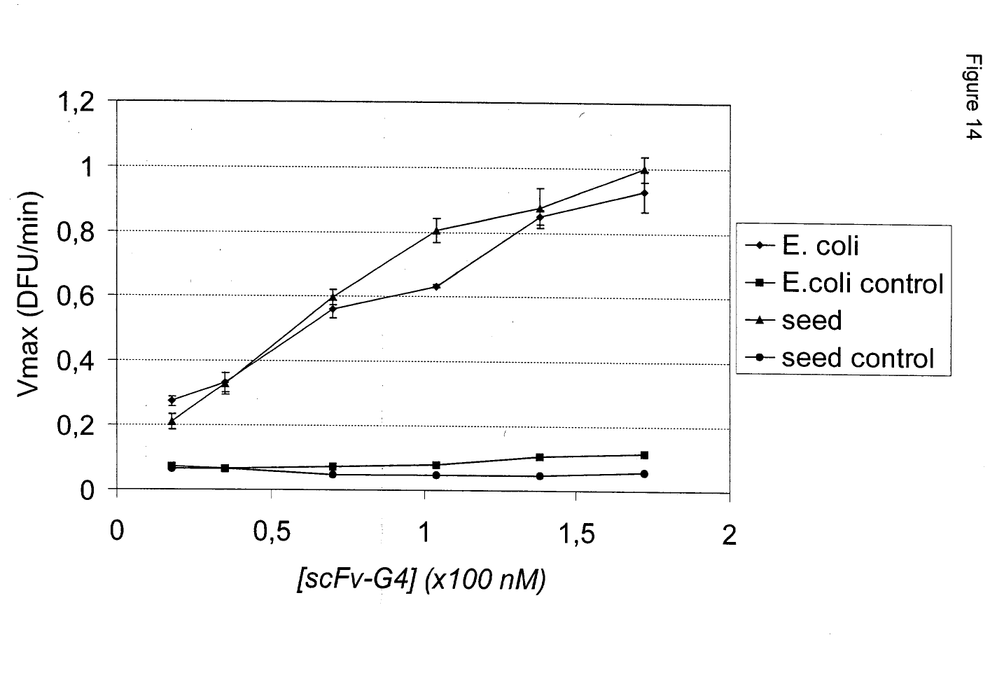

Antigen-binding activity of seed extracted G4-proteins was measured and compared with E. coli-extracted G4 by ELISA. We used seed extract of the F38 ‘phas’-seed stock containing 36.5% G4 (table 4). Different amounts of scFv-G4 were incubated with excess antigen, dihydroflavonole-4-reductase, with bound antigen measured by sandwich-ELISA (FIG. 13). ELISA-signal curves were set up for both the bacterial and plant produced scFv and compared. The curves overlap each other (FIG. 14), indicating that the plant-produced and bacterial-produced scFv have similar antigen-binding activity per mg protein.

### Example 5

### Transformation of Phaseolus acutifolius and scFv Accumulation in Transgenic Segregating Bean Seeds

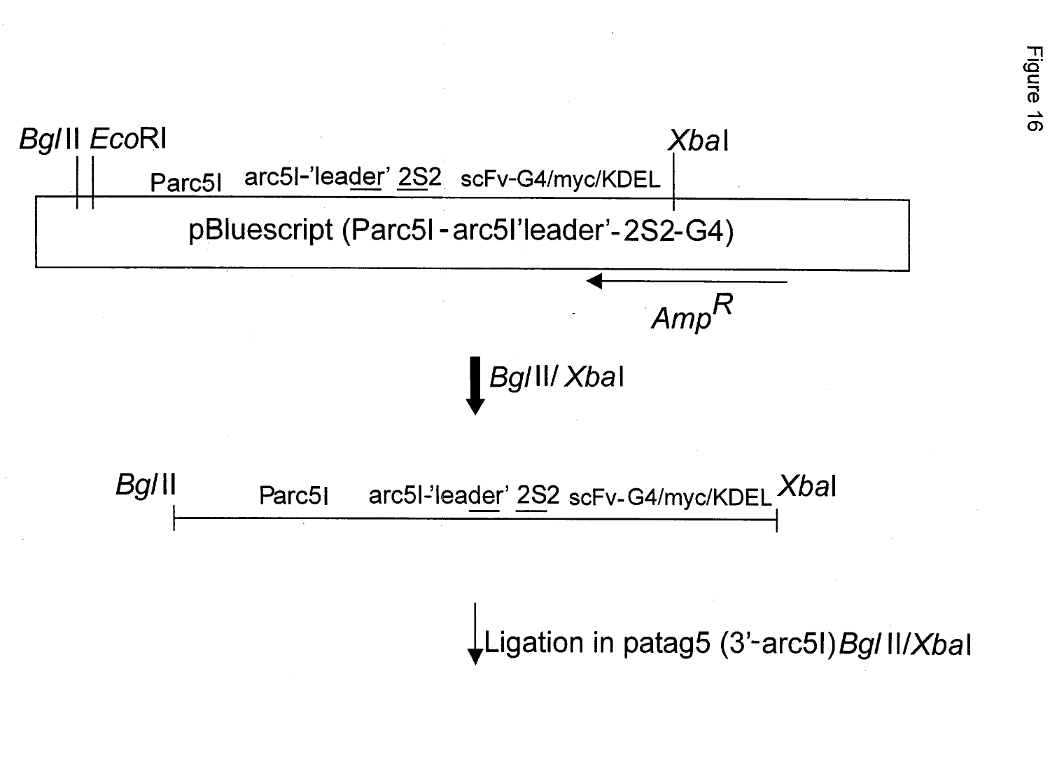

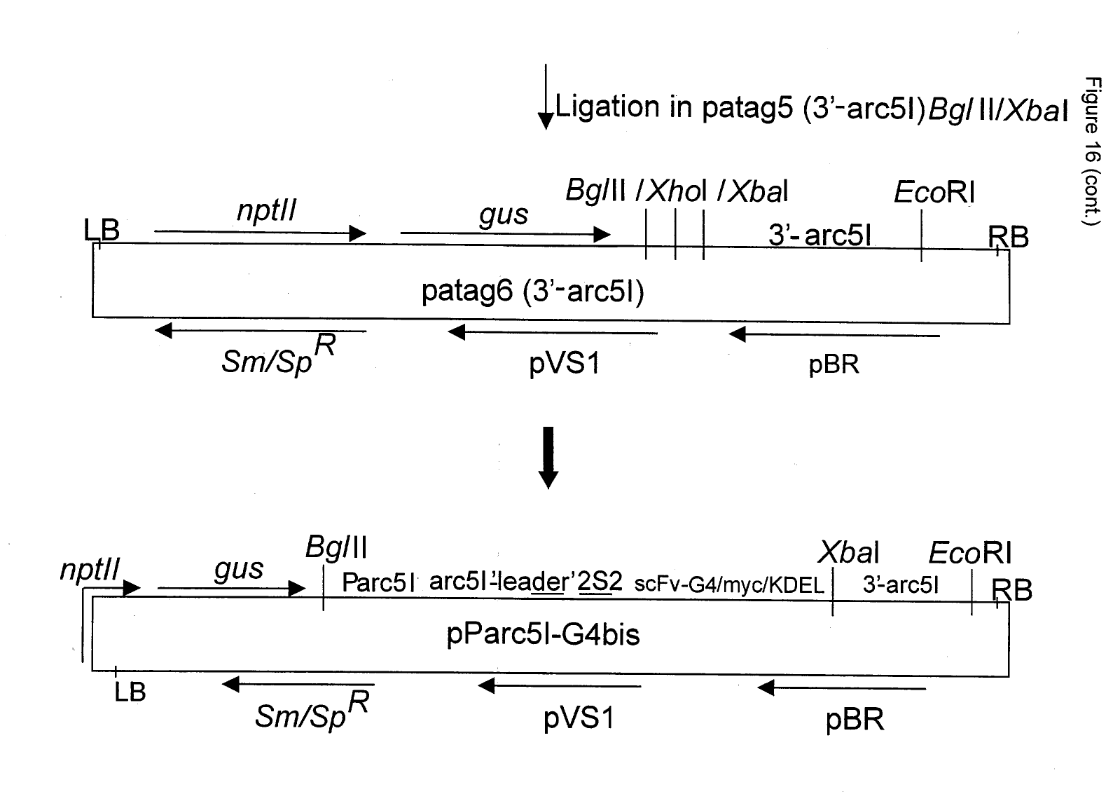

Phaseolus acutifolius TB1 Was transformed with pParc5I-G4bis (FIG. 16). pParc5I-G4bis contains the same T-DNA as pParc5I-G4, except that it contains an additional P35S-GUS-construct for segregation analysis of transgenic plants.

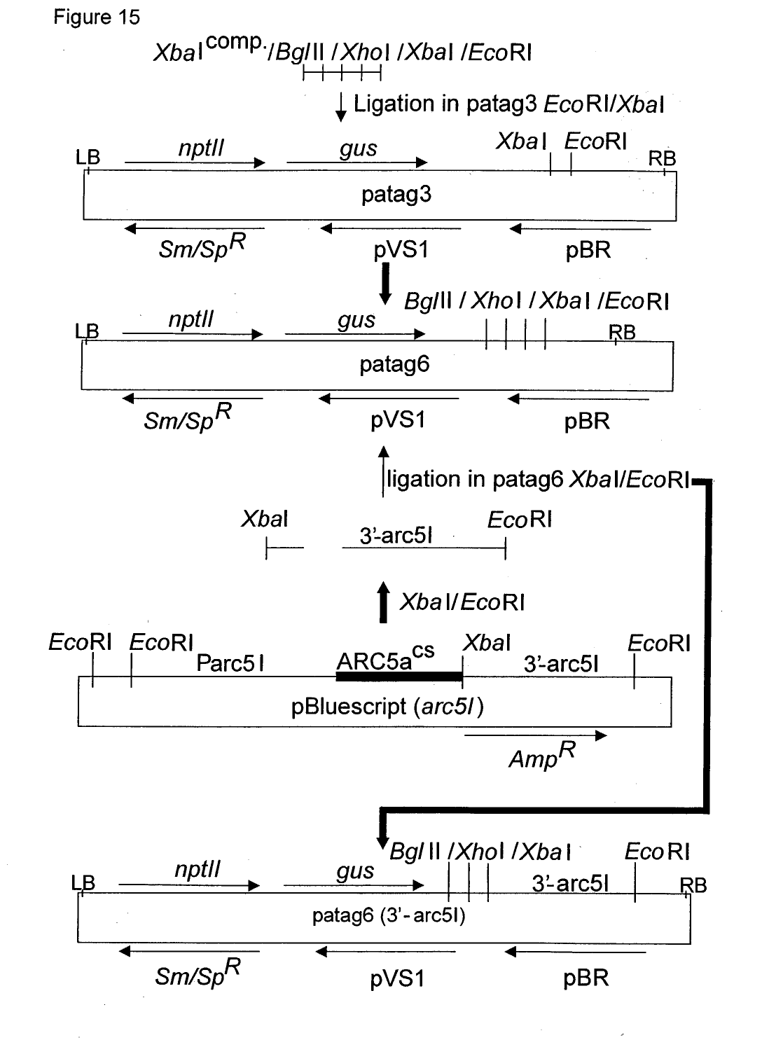

For the construction of this T-DNA vector, we first made the pilot construct patag6 (3′-arc5I) (FIG. 15) according to the cloning step procedure for patag5 (3′-arc5I) (FIG. 3). An oligonucleotide was made, which inserts a few unique restriction sites in the T-DNA-vector patag3 (Goossens et al., 1999). The oligonucleotide contained the following restriction sites from its 5′-end (proximal to the Xba I site) to its 3′-end (proximal to the Eco RI site): the 5′-‘sticky’ end of Eco RI, the restriction sites Xba I, Xho I, and Bgl II and a 3′-‘sticky’ end that complements Xba I, but does not regenerate the site after ligation. The oligonucleotide was ligated into patag3 digested with Eco RI and Xha I. This resulted in the vector patag6. The sequence of the inserted oligonucleotide was checked and a clone with the correct insert was selected for further cloning steps. From the vector pBluescript (arc5I) (Goossens et al., 1995), which contains the genomic sequence of the arc5I-gene, we cut out the 3′-expression signals of arc5I (3′-arc5I) using Xba I and Eco RI. The 3′-arc5I-fragment was then ligated into patag6 digested with the same restriction enzymes. This resulted in the vector patag6 (3′-arc 5I). This pilot construct was used to make pParc5I-G4bis (FIG. 16). The arc5I-‘leader’ and the G4-coding sequence was cut out of the vector pBluescript (Parc5I-arc 5‘leader’-2S2-G4) (FIG. 6) using the restriction sites Bgl II and Xba I, then ligated into the patag6 (3′-arc5I) vector digested with Bgl II and Xba I. This resulted in the T-DNA vector pParc5I-G4bis.

Three transgenic plants were obtained by using the protocol of Dillen et al. (1997). As the three plants were regenerated from the same callus, it was expected that they were clones from the same transformation event. Seeds were collected and protein extracts were made from separate seeds using the same buffer as used in the Arabidopsis seed extraction and according to the method of Goossens et al. (1999) as described above. Total soluble protein concentration was measured spectrophotometrically at 280 nm and G4 accumulation was determined by quantitative Western blot.

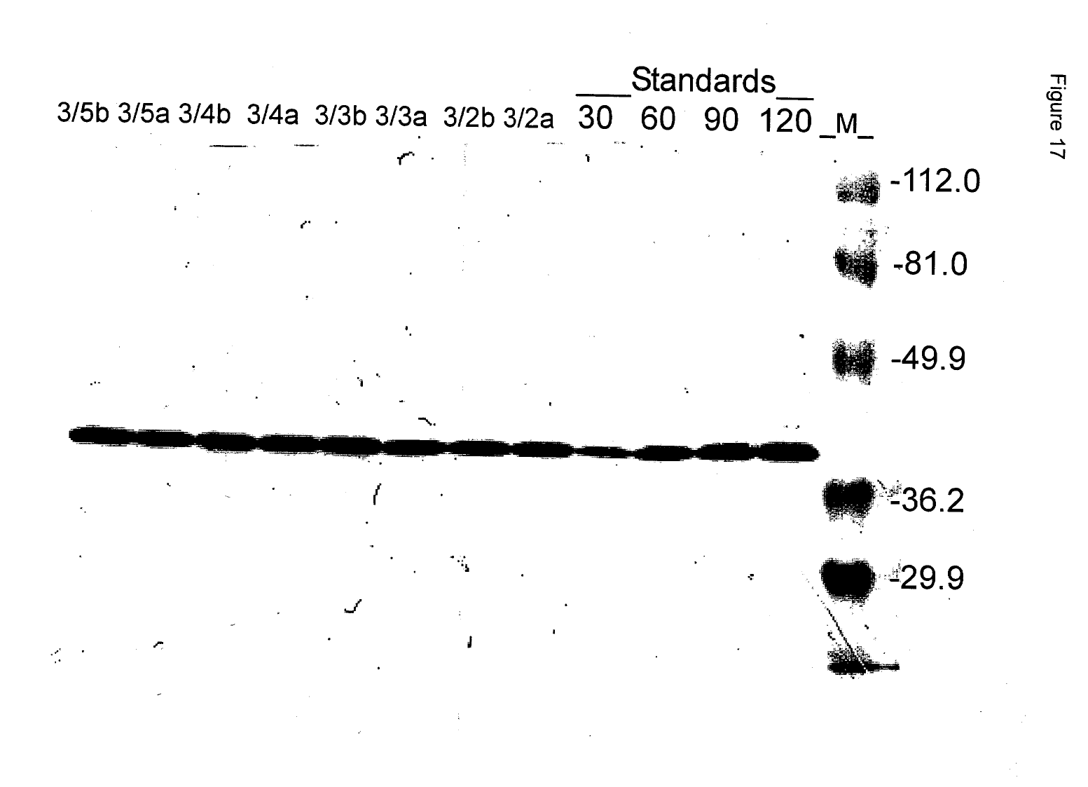

G4 was detected as a single protein band (FIG. 17) in, on average, 3 of 4 seeds for all three segregating seed stocks. Therefore, these transformants most probably contain a single T-DNA-locus. All analyzed G4-accumulating seeds contained 2-2.5% G4, relative to total soluble protein, or 2-2.5 milligrams scFv per gram fresh weight seed.

So far, only one paper reported scFv production in leguminous species. Perrin et al. (2000) obtained 9 micrograms scFv per gram fresh weight in pea seeds with the legA promoter. Thus, the accumulation with the arc5I promoter construct is 2 to 3 hundred times higher than the reported levels with the legA promoter. As we only obtained one transgenic plant line, we believe that plant lines with even higher scFv levels can be obtained. Goossens et al. (1999) obtained 4× higher levels of ARC5I protein in homozygous seed stocks compared to segregating seed stocks, using the complete arc5I gene, including its promoter. As such, after obtaining more transgenic lines and selection of homozygous lines, we expect to reach at least 10% scFv levels in Phaseolus acutifolius, by using the arc5I promoter construct.

The previous examples are provided to illustrate the present inventions and are not intended to limit the scope of the claimed inventions. Other variants of the inventions will be readily apparent to those of ordinary skill in the art and are encompassed by the present invention.

### REFERENCES

All publications, patents, patent applications and other references cited herein are hereby incorporated by this reference in their entirety.

Bustos, M. M., Begum, D., Kalkan, F. A., Battraw, M. J., and Hall, T. C. (1991). Positive and negative cis-acting DNA domains are required for spatial and temporal regulation of gene expression by a seed storage promoter. EMBO 10 (6), 1469-1479.

Clough, S. J., and Bent, A. F. (1998). Floral dip: a simplified method for Agrobacterium-mediated transformation of Arabidopsis thaliana. Plant J. 16 (6), 735-743.

De Jaeger, G., Buys, E., Eeckhout, D., De Wilde, C., Jacobs, A., Kapila, J., Angenon, G., Van Montagu, M., Gerats, T., and Depicker, A. High level accumulation of single-chain variable fragments in the cytosol of transgenic Petunia hybrida (1999). Eur. J. Biochem. 259, 426-434.

Denecke, J., De Rycke, R. and Bottermnan, J. (1992). Plant and mammalian sorting signals for protein retention in the endoplasmic reticulum contain a conserved epitope. EMBO J 11: 2345-2355.

Dillen, W., De Clercq, J., Goossens, A., Van Montagu, M. and Angenon, G. (1997). Agrobacterium-mediated transformation of Phaseolus acutifolius A. Gray. Theor. Appl. Genet. 94, 151-158.

Evan, G. I., Lewis, G. K., Ramsay, G., and Bishop, J. M. (1985). Isolation of monoclonal antibodies specific for Human c-myc Proto-oncogene product. Mol. And Cell. Biol. 12, 3610-3616.

Fiedler, U., Phillips, J. Artsaenko, 0. and Conrad, U. (1997). Optimization of scFv antibody production in transgenic plants. Immunotechnology 3, 205-216

Goddijn, O. J. M. and Pen, J. (1995). Plants as bioreactors. Trends Biotechnol. 13, 379-387.

Goossens, A., Geremia, R., Bauw, G., Van Montagu, M. And Angenon, G. (1994). Isolation and characterization of arcelin-5 proteins and cDNAs. Eur. J. Biochem. 225, 787-795.

Goossens, A., Ardiles Diaz, W., De Keyser, A., Van Montagu, M., and Angenon, G. (1995). Nucleotide sequence of an arcelin 5-I genomic clone from wild Phaseolus vulgaris (Z50202). Plant Physiol. 109, 722, PGR95-075.

Goossens, A., Dillen, W., De Clercq, J., Van Monatgu, M., and Angenon, G. (1999). The arcelin-5 gene of Phaseolus vulgaris directs high seed-specific expression in transgenic Phaseolus acutifolius and Arabidopsis plants. Plant Physiology, Vol. 120, p. 1095-1104.

Hemming, D. (1995) Molecular farming: using transgenic plants to produce novel proteins and other chemicals. AgBiotech News Inform. 7, 19N-29N.

Krebbers, E., Herdies, L., De Clercq, A., Seurinck, J., Leemans, J., Van Damme, J., Segura, M., Gheysen, G., Van Montagu, M. and Vandekerckhove, J. (1988). Determination of the processing sites of an Arabidopsis 2S albumin and characterization of the complete gene family. Plant Physiol 87: 859-866.

Perrin, Y., Vaquero, C., Gerrard, I., Sack, M., Drossard, J., Stöger, E., Christou, P., and Fischer, R. (2000). Transgenic pea seeds as bioreactors for the production of a single-chain Fv fragment (scFV) antibody used in cancer diagnosis and therapy. Molecular breeding 6, 345-352.

Valvekens, D., Van Montagu, M, and Van Lijsebettens, M. (1988). Agrobacterium tumefaciens-mediated transformation of Arabidopsis thaliana root explants by using kanamycin selection. PNAS USA 85, 5536-5540.

van der Geest, A. H. M., Hall, G. E., Spiker, S., and Hall, T. C. (1994). The β-phaseolin gene is flanked by matrix attachment regions. The Plant Journal 6(3), 413-423.

Vitale, A. and Bollini, R. (1995) Legume storage proteins. In Seed development and germination (Kigel, J. and Galili, G. eds.) p73-102, Marcel Dekker Inc., New York.

Whitelam, G. C., Cockburn, B., Gandecha, A. R. and Owen, M. R. L. (1993). Heterologous protein production in transgenic plants. Biotechnol. Genet. Eng. Rev. 11, 1-29.

What is claimed is:

1. A seed preferred expression cassette having gene regulatory elements comprising:
a) a promoter comprising an arcelin promoter of sequence of SEQ ID NO: 1 or a phaseolin promoter of SEQ ID NO: 5;
b) a leader comprising an arcelin 5I leader sequence of SEQ ID NO: 2 or a TMV omega leader; and
c) an arcelin 5I 3′ end comprising the sequence of SEQ ID NO: 3.

2. The seed preferred expression cassette of claim 1 wherein the promoter is the phaseolin promoter of SEQ ID NO: 5.

3. The seed preferred expression cassette of claim 1 wherein the leader is a TMV omega leader.

4. The seed preferred expression cassette of any one of claims 1-3, further comprising the sequence shown in SEQ ID NO: 4, encoding the 2S2 storage albumin signal peptide.

5. The seed preferred expression cassette of any one of claims 1-4, further comprising a gene of interest placed between the leader sequence and the arcelin 5I 3′ sequence.

6. The seed preferred expression cassette of claim 5, wherein said gene of interest is fused in frame to the sequence encoding the 2S2 storage albumin signal peptide.

7. The seed preferred expression cassette of claim 5 or claim 6, wherein the gene of interest encodes a single chain Fv fragment.

8. A process for obtaining seed preferred expression of a heterologous protein at a level of at least about 10% of the total soluble seed protein, wherein said heterologous protein is not an unmodified seed storage protein, said process comprising:
introducing into a plant or plant cell a gene of interest encoding a heterologous protein;
growing said plant or plant cell; and
expressing a heterologous protein at a level of at least about 10% of the total soluble seed protein, provided that said heterologous protein is not an unmodified seed storage protein.

9. The process of claim 8, wherein the level is at least 15% of the total soluble seed protein.

10. The process of claim 8 or claim 9, wherein the process involves using a seed preferred expression cassette having gene regulatory elements comprising:
a) a promoter comprising an arcelin promoter of sequence of SEQ ID NO: 1 or a phaseolin promoter of SEQ ID NO: 5;
b) a leader comprising an arcelin 5I leader sequence of SEQ ID NO: 2 or a TMV omega leader; and
c) an arcelin 5I 3′ end comprising the sequence of SEQ ID NO: 3.

11. A plant cell transformed with the seed preferred expression cassette of any one of claims 1-7.

12. A transgenic plant comprising the seed preferred expression cassette of any one of claims 1-7.

13. The seed preferred expression cassette of claim 2 further comprising SEQ ID NO: 4.

14. The seed preferred expression cassette of claim 3 further comprising SEQ ID NO: 4.

15. The seed preferred expression cassette of claim 2, wherein a gene of interest is placed between said leader sequence and said arcelin 5I 3′ sequence.

16. The seed preferred expression cassette of claim 3, wherein a gene of interest is placed between said leader sequence and said arcelin 5I 3′ sequence.

17. The seed preferred expression cassette of claim 4, wherein a gene of interest is placed between said leader sequence and said arcelin 5I 3′ sequence.

18. The seed preferred expression cassette of claim 13, wherein a gene of interest is placed between said leader sequence and said arcelin 5I 3′ sequence.

19. The seed preferred expression cassette of claim 14, wherein a gene of interest is placed between said leader sequence and said arcelin 5I 3′ sequence.

20. The seed preferred expression cassette of claim 2, further comprising a gene of interest is fused in frame to a sequence encoding the 2S2 storage albumin signal peptide.

21. The seed preferred expression cassette of claim 3, further comprising a gene of interest is fused in frame to a sequence encoding the 2S2 storage albumin signal peptide.

22. The seed preferred expression cassette of claim 15, wherein the gene of interest encodes a single chain Fv fragment.

23. The seed preferred expression cassette of claim 16, wherein the gene of interest encodes a single chain Fv fragment.

24. The seed preferred expression cassette of claim 17, wherein the gene of interest encodes a single chain Fv fragment.

25. The process of claim 8, wherein expressing a heterologous protein comprises utilizing an expression cassette comprising:
a) a phaseolin promoter comprising the sequence of SEQ ID NO: 5;
b) an arcelin 5I leader comprising the sequence of SEQ ID NO: 2;
c) an arcelin 5I 3′ end comprising the sequence of SEQ ID NO: 3; and
d) a gene of interest placed between said arcelin 5I leader sequence and said arcelin 5I 3′ sequence.

26. The process of claim 8, wherein expressing a heterologous protein comprises utilizing an expression cassette comprising:
a) an arcelin promoter comprising the sequence of SEQ ID NO: 1;
b) a TMV omega leader;
c) an arcelin 5I 3′ end comprising the sequence of SEQ ID NO: 3; and
d) a gene of interest placed between said TMV omega leader sequence and said arcelin 5I 3′ sequence.

27. The process of claim 8, wherein expressing a heterologous protein comprises utilizing an expression cassette comprising:
a) a phaseolin promoter comprising the sequence of SEQ ID NO: 5;
b) an arcelin 5I leader comprising the sequence of SEQ ID NO: 2;
c) a 2S2 storage albumin signal peptide comprising the sequence of SEQ ID NO: 4;
d) an arcelin 5I 3′ end comprising the sequence of SEQ ID NO: 3; and
e) a gene of interest placed between said arcelin 5I leader sequence and said arcelin 5I 3′ sequence.

28. The process of claim 8, wherein expressing a heterologous protein comprises utilizing an expression cassette comprising:
a) an arcelin promoter comprising the sequence of SEQ ID NO: 1;
b) a TMV omega leader;
c) a 2S2 storage albumin signal peptide comprising the sequence of SEQ ID NO: 4;
d) an arcelin 5I 3′ end comprising the sequence of SEQ ID NO: 3; and
e) a gene of interest placed between said a TMV omega leader sequence and said arcelin 5I 3′ sequence.

29. The process of claim 9, wherein expressing a heterologous protein comprises utilizing an expression cassette comprising:
a) an arcelin promoter comprising the sequence of SEQ ID NO: 1;
b) an arcelin 5I leader comprising the sequence of SEQ ID NO: 2;
c) an arcelin 5I 3′ end comprising the sequence of SEQ ID NO: 3; and
d) a gene of interest placed between said arcelin 5I leader sequence and said arcelin 5I 3′ sequence.

30. The process of claim 9, wherein expressing a heterologous protein comprises utilizing an expression cassette comprising:
a) a phaseolin promoter comprising the sequence of SEQ ID NO: 5;
b) an arcelin 5I leader comprising the sequence of SEQ ID NO: 2;
c) an arcelin 5I 3′ end comprising the sequence of SEQ ID NO: 3; and
d) a gene of interest placed between said arcelin 5I leader sequence and said arcelin 5I 3′ sequence.

31. The process of claim 9, wherein expressing a heterologous protein comprises utilizing an expression cassette comprising:
a) an arcelin promoter comprising the sequence of SEQ ID NO: 1;
b) a TMV omega leader;
c) an arcelin 5I 3′ end comprising the sequence of SEQ ID NO: 3; and
d) a gene of interest placed between said TMV omega leader sequence and said arcelin 5I 3′ sequence.

32. The process of claim 9, wherein expressing a heterologous protein comprises utilizing an expression cassette comprising:
a) a phaseolin promoter comprising the sequence of SEQ ID NO: 5;
b) an arcelin 5I leader comprising the sequence of SEQ ID NO: 2;
c) an arcelin 5I 3′ end comprising the sequence of SEQ ID NO: 3;
d) a 2S2 storage albumin signal peptide comprising the sequence of SEQ ID NO: 4; and
e) a gene of interest placed between said arcelin 5I leader sequence and said arcelin 5I 3′ sequence.

33. The process of claim 9, wherein expressing a heterologous protein comprises utilizing an expression cassette comprising:
a) an arcelin promoter comprising the sequence of SEQ ID NO: 1
b) a TMV omega leader;
c) an arcelin 5I 3′ end comprising the sequence of SEQ ID NO: 3;
d) a 2S2 storage albumin signal peptide comprising the sequence of SEQ ID NO: 4; and
e) a gene of interest placed between said TMV omega leader sequence and said arcelin 5I 3′ sequence.

34. A plant comprising a plant cell transformed with the seed preferred expression cassette of claim 2.

35. A plant comprising a plant cell transformed with the seed preferred expression cassette of claim 3.

36. A transgenic plant comprising the seed preferred expression cassette of claim 2.

37. A transgenic plant comprising the seed preferred expression cassette of claim 3.
